(19) United States
(12) Patent Application Publication
(10) Pub. No.: US 2026/0055152 A1
(43) Pub. Date: Feb. 26, 2026

(54) INTRACELLULAR CX3CL1 DOMAIN PEPTIDES AND USES THEREOF

(71) Applicant: University of Connecticut, Farmington, CT (US)

(72) Inventors: Riqiang YAN, Farmington, CT (US); Manoshi GAYEN, New Britain, CT (US); Wanxia HE, Farmington, CT (US)

(21) Appl. No.: 19/103,188

(22) PCT Filed: Aug. 31, 2023
(86) PCT No.: PCT/US2023/031695

§ 371 Date: Feb. 11, 2025

Related U.S. Application Data
(60) Provisional application No. 63/402,608, filed on Aug. 31, 2022.

(51) Int. Cl. C07K 14/52; A61K 38/00; A61P 25/28

(52) U.S. Cl. CPC C07K 14/521; A61P 25/28; A61K 38/00; C07K 2319/10

(57) ABSTRACT

Described is a polypeptide including the intracellular domain of CX3CL1 (CX3CL1-ICD), including an amino acid sequence that is at least 90% identical to SEQ ID NO: 1 and pharmaceutical compositions including the above-described polypeptide and a pharmaceutically acceptable excipient. Also included are nucleic acids expressing the polypeptides and expression cassettes and vectors comprising the nucleic acids. The polypeptides can be used in methods of treating a subject exhibiting a symptom of a neurodegenerative disease and/or diagnosed with a neurodegenerative disease and methods of treating a subject exhibiting a symptom of diabetes and/or diagnosed with diabetes comprising administering to the subject the pharmaceutical composition described herein.

### CROSS-REFERENCE TO RELATED APPLICATIONS

This application claims priority to U.S. Provisional Application Ser. No. 63/402,608, filed on Aug. 31, 2022, which is incorporated herein by reference in its entirety.

### GOVERNMENT SUPPORT

This invention was made with government support under AG046929, NS074256, AG058261, and AG025493 awarded by the National Institutes of Health. The government has certain rights in the invention.

### SEQUENCE LISTING

The instant Application contains a Sequence Listing which has been submitted electronically in ST.26 XML format and is hereby incorporated by reference in its entirety. Said XML copy, created on Aug. 29, 2023 is named “UCT0321PCT sequence listing1 Aug. 29, 2023.XML” and is 16,384 bytes in size.

### FIELD OF THE DISCLOSURE

The subject disclosure relates to peptides from the intracellular CX3CL1 domain and uses thereof in regulating insulin and insulin growth factor signaling for diabetes, anti-aging, neuroprotection, and neurodegeneration.

### BACKGROUND

The type-1 transmembrane chemokine CX3CL1, also known as fractalkine, is known to exert a signaling function by binding to its cognate receptor CX3CR1. In the brain, CX3CL1 is largely expressed by neurons, while its receptor CX3CR1 is predominantly expressed by microglia. This ligand-receptor interaction in the brain triggers neuron-microglia crosstalk by altering neuroinflammatory responses and causing neurotoxic or neuroprotective effects depending on various neurological diseases. How CX3CL1 exerts its effects on neuroprotection is not well understood.

What is needed in the art are active fragments of CX3CL1 that provide new therapeutic uses.

### SUMMARY

In an aspect, disclosed is a polypeptide comprising an amino acid sequence that is at least 90% identical to SEQ ID NO: 1.

In another aspect, disclosed is a pharmaceutical composition comprising the above-described polypeptide and a pharmaceutically acceptable excipient.

Also disclosed are nucleic acids expressing the polypeptides and expression cassettes and vectors comprising the nucleic acids.

In yet another aspect, disclosed is a method of treating a subject exhibiting a symptom of a neurodegenerative disease and/or diagnosed with a neurodegenerative disease, the method comprising administering to the subject the pharmaceutical composition described herein.

In still another aspect, disclosed is a method of treating a subject exhibiting a symptom of diabetes and/or diagnosed with diabetes, the method comprising administering to the subject the pharmaceutical composition described herein.

These and other aspects are described in more detail below.

### BRIEF DESCRIPTION OF THE DRAWINGS

Reference will now be made to the accompanying drawings, which are not necessarily drawn to scale.

FIGS. 1A-D: Designing neuron-specific synthetic CX3CL1-ICD peptides. FIG. 1A shows a schematic representation of the synthetic Tet34 (SEQ ID NO: 3), derived from CX3CL1-ICD, and Tet34s peptides (SEQ ID NO: 4). FIG. 1B shows mouse neuroblastomas-2a (N2A) cells were treated with synthetic Alexa Fluor®-488-Tet34 peptides for 24 hrs, which contains an Alexa Fluor®-488 tag at the terminus of Tet34, followed by fixation at different time points. Alexa Fluor®-488-Tet34 is retained within cells for up to 72 hrs, whereas scrambled peptide Tet34s was much less detected in N2A cells. Scale bar=10 μm. FIG. 1C shows peptide Tet34 is selectively uptaken by neurons when the mixed primary brain culture was treated with peptides for 12 hrs. Scrambled peptide was not readily detected in the mixed primary culture. Scale bar=20 μm. FIG. 1D shows EDU uptake kinetics of N2A, treated with indicated concentrations of Tet34 or Tet34s at 12 hrs, 24 hrs and 36 hrs after treatment, were plotted (N=3. ***P<0.001, t-student test).

FIGS. 2A-B: CX3CL1-ICD peptides induce insulin/IGF-1 signaling pathways. FIG. 2A shows protein lysates from mouse neuroblastoma-2a (N2A) cells, treated with Tet34 or controls, were examined by the indicated antibodies. FIG. 2B shows Bar graphs compared levels between Tet34 treatment group vs Tet34s or Tet34 vs control. Tet34 treatment for 24 or 48 hrs significantly differentially upregulated the phosphorylation levels of key mediators in the insulin/IGF pathway (N=3 experiments; * P<0.05; ** P<0.01;*** P<0.001, one-way ANOVA).

FIGS. 3A-B: CX3CL1-ct overexpression in transgenic mice induces insulin/IGF-1 signaling pathways. FIG. 3A shows neuron-specific overexpression of CX3CL1-ct in mice was achieved by breeding CX3CL1-ct mice with CaMKIIa-Tet mice. The transgene was turned on by doxycycline withdrawal at P45. Hippocampal and cortical protein lysates from the indicated genotypes of mice were examined with indicated antibodies by western blotting. CX3CL1-ct/tTa mice had significantly increased expression of insulin receptor (anti-IGFβ) and insulin growth factor-1 (IGF-1) receptor (IGF-1R; detected by anti-IGF-1Rβ). Downstream molecules insulin substrate 1 (IRS1) and IRS2, Akt, PDK1 and Foxo3 were more obviously activated. FIG. 3B shows bar graphs show comparative levels normalized to the loading control. N=3 independent experiments (*P<0.05; **P<0.01; ***P<0.001, one-way ANOVA).

FIGS. 4A-H: CX3CL1-ICD induces expression of genes for proliferation. FIGS. 4A, 4B, 4C, and 4D show that significant overexpression of neuronal differentiation markers ASCL1 and NeuroD1 as well as cell cycle regulators such as Cyclin, Cdk, and PCNA was observed in CX3CL1-ct/tTa mice, using lysates prepared the same as in FIG. 3A and Figure B. Sox2, Sox5, Sox8 and Sox9 were also elevated and shown in FIG. 4C. FIG. 4B and FIG. 4D show bar graphs show comparative protein expression levels normalized to the loading control. N=3 independent experiments (*P<0.05; **P<0.01; ***P<0.001, one-way ANOVA). FIG. 4E and FIG. 4G show the same set of protein levels was compared on the western blots using protein lysates from N2A cells treated with Tet34 or Tet34s for 24 or 48 hrs. FIG. 4F and FIG. 4H show bar graphs show protein expression levels normalized to the loading control. N=3 independent experiments (*P<0.05; **P<0.01; one-way ANOVA).

FIGS. 5A-B: CX3CL1-ICD peptides represses the apoptotic pathway. FIG. 5A shows N2A cells treated with Tet34 or Tet34s were prepared for examined by the western blotting experiment with the indicated antibodies to p53, Bax, p27 and p21. FIG. 5 shows bar graphs show protein expression levels normalized to the loading control β-actin. N=3 independent experiments (*P<0.05; **P<0.01; one-way ANOVA).

FIGS. 6A-F: CX3CL1-ICD peptide confers protection against amyloid beta-induced stress. FIG. 6A shows the results of N2A cells treated with 5 μM CX3CL1-ICD peptides for 36 hrs. A subset of the cells was treated with amyloid beta at 24 hrs post-treatment with Tet-CX3CL1-ICD peptides for 12 hrs prior to evaluation of stress-induced markers. FIG. 6C shows thatTet34 significantly attenuated mitochondrial apoptotic markers and FIG. 6E ameliorated ER stress-induced markers. FIG. 6B, FIG. 6D, and FIG. 6F are bar graphs show protein expression levels normalized to the loading control. n=3 independent experiments (n=3 experiments; *P<0.05; **P<0.01; ***P<0.001, one-way ANOVA; error bars indicate ±SEM).

FIG. 7 illustrates the mechanism of modulation of insulin or insulin growth factor-1 (IGF).

FIGS. 8A-D: In vivo intranasal Tet34 delivery induces TGFβ and insulin signaling pathway in both cortex and Hippocampus. FIG. 8A shows the results of 2 μM Tet34 or scrambled Tet34s reconstituted in 20 μl saline and instilled intranasally in anesthetized mice via a handheld pipette over a period of 30 minutes. 24-hour post-drug delivery, the cortex and hippocampus were collected, and lysates were immunoprobed for TGFβ and insulin signaling pathway. FIG. 8B, FIG. 8C, and FIG. 8D are bar graphs that show that Tet34 significantly increased TGFβ expression (8B) and phosphorylated levels of IRS-1 (FIG. 8D) and AKT (FIG. 8C). (N=3 experiments; *P<0.05; **P<0.01; ***P<0.001, one-way ANOVA).

FIGS. 9A-D: Peptide Alexa Fluor®-488-Tet34 is preferentially taken up by Map2-marked neurons. Primary mixed brain co-cultures were treated with Alexa Fluor®-488-Tet34 or Alexa Fluor®-488-Tet34s peptides for 12 hrs. Cells were marked by Map2 for neurons, Iba1 for microglia and Olig2 for oligodendrocytes. Images were taken on a Zeiss confocal microscope using 63× or 20× objectives. Scale bar=20 um (63×) and 50 μm (20×). Co-immunofluorescence quantification was conducted by measuring Alexa Fluor®-488-pixel intensity within Map2-, Gfap-, and Iba1-positive cell bodies using ImageJ. N=61 neurons, 56 astrocytes, 42 microglia and 19 oligodendrocytes (***P<0.001, comparing only to neurons; student's t test). FIG. 9A quantification showed intensity co-stained with Alexa Fluor®-488-Tet34 with Map2-marked neurons, or Gfap-marked astrocytes or Iba1-marked microglia. FIG. 9B, FIG. 9C, and FIG. 9D show confocal staining of mixed cultures with indicated antibodies for microglia (FIG. 9B), oligodendrocytes (FIG. 9C), neurons and astrocytes (FIG. 9D). Scale bar is 50 μm (FIG. 9B and FIG. 9C) and 20 μm (FIG. 9D).

FIGS. 10A-B: FIG. 10A shows that CX3CL1-ICD-derved Tet34 peptide induces the TGFβ/Smad signaling pathway in N2A cells at 24 hrs and 48 hrs post-treatment in N2A cells. FIG. 10B shows bar graphs show that Tet34 significantly increased TGFβ expression and phosphorylated Smad levels at 48 hrs post-treatment. (N=3 experiments; *P<0.05; **P<0.01; ***P<0.001, one-way ANOVA). Induced expression of TGFβ signaling in transgenic mice expressing CX3CL1-ct.

FIGS. 11A-C: CX3CL1-ICD peptides attenuate Foxo activity via IGF1-Rβ/Akt pathway. FIG. 11A shows Western blot analyses from N2A cultures treated with Tet-CX3CL1-ICD peptides in presence or absence of Akt inhibitor, MK2206. Treatment with MK2206 abolished the Tet34 induced effects on Foxo3 phosphorylation. FIG. 11B and Figure C show bar graphs that show protein expression levels normalized to the loading control calnexin. N=3 independent experiments (*P<0.05; **P<0.01; one way ANOVA).

FIGS. 12A-B: Overexpressed neuronal CX3CL1-ct reduces the pro-apoptotic genes. FIG. 12A shows Western blot analyses from hippocampal lysates of CX3CL1-ct/tTa mice showed significantly down-regulated expression of p53, Bax, p27 and p21. FIG. 12B shows bar graphs showing protein expression levels normalized to the loading control calnexin. N=3 independent experiments (*P<0.05; **P<0.01; one way ANOVA).

FIGS. 13A-C: CX3CL1-ICD peptides attenuate apoptosis induced by hydrogen peroxide. FIG. 13A are Western blot analyses from N2A cultures treated with Tet34 or Tet34s peptides in presence either 2% serum or hydrogen peroxide. Tet34 attenuated the expression of apoptotic markers activated by hydrogen peroxide. FIG. 13B and FIG. 13C are bar graphs that show protein expression levels normalized to the loading control calnexin. N=3 independent experiments (*P<0.05; **P<0.01; one way ANOVA).

FIGS. 14A-D: CX3CL1-ICD peptides attenuate apoptosis induced by hydrogen peroxide. N2A cells were pretreated with Tet34 or Tet34s peptides for 24 hrs followed by hydrogen peroxide treatment (FIG. 14A, FIG. 14B, FIG. 14C, and FIG. 14D). Apoptotic and live cell populations were analyzed at different time points by flow cytometry. N=3 independent experiments (*P<0.05; **P<0.01; Student's t test).

FIGS. 15A-B: Neuronal overexpression of CX3CL1-ct upregulates IGF-1Rβ mRNA in the hippocampus of transgenic mice. Messenger RNA was isolated from different regions of the brain and used for real time PCR quantification of IGF-1Rβ (FIG. 15A) and IGF1 (FIG. 15B) mRNA. N=4-5 animals (*P<0.05; **P<0.01; Student's t test).

FIG. 16: Illustration of CX3CL1-ct vs. CX3CL1. The intracellular domain (CX3CL1-ICD) is released after γ-secretase cleavage.

FIG. 17: Diagram showing phenotypic changes in PS19 mice described in the art. Formation of neurofibrillary tangles is relying on the expression of mutant Tau in this mouse model, and increased Tau phosphorylation correlated with Tau protein aggregation and formation of neurofibrillary tangles.

FIG. 18: Expression of CX3CL1-ct decreases levels of phosphorylated Tau in PS19 mice. Brain lysates were prepared from mice for Western blotting experiments and phosphorylated tau proteins were detected by the indicated antibodies. Phosphorylation of Tau at Thr231, Thr181, Thr205, Ser396 and S404 was reduced in both male and female Tg-CX3CL1/tTA/PS19 mice, when compared to control littermates of PS19 mice. Data are presented from 3 pairs of mice (*p<0.05; **p<0.01, Student t test).

FIG. 19: Expression of CX3CL1-ct decreases Tau aggregation in PS19 mice. The brain tissues were homogenized in RIPA buffer, which contains detergents 1% NP-40 and 0.1% SDS, followed by sonication on ice. The RIPA soluble protein fractions were then collected from the supernatant after centrifugation at 21400 g×30 min. The pellet was then given a second RIPA wash followed by centrifugation to remove all soluble protein fractions. The pellet formed after the second RIPA wash was resuspended in urea buffer (50 mM Tris-HCl pH 8.5, 8M urea, 2% SDS) for 30 min at room temperature to acquire the RIPA-insoluble protein fraction. These fractions were used for western blot analysis of proteins detected by the indicated antibodies. Soluble fractions contain Tau monomers or soluble oligomers while insoluble fractions contain mostly Tau aggregates that are likely in the form of neurofibrillary tangles. It is noted that pTau S199 and S404 were detectable in control mice (WT, CamkII-tTA and Tg-Cx3CL1-ct mice), but not the phosphorylated Tau at Thr181, Thr231. Ps19 mice showed high levels in all these sites. Strikingly, overexpressing Cx3CL1-ct in PS19 mice (Tg-CX3CL1-ct/tTA/PS19 mice) caused significant reduction in all these p-Tau levels. Mutant Tau migrated slower than endogenous Tau on the western blots.

FIG. 20: Expression of CX3CL1-ct in PS19 mice improve learning and memory behaviors. Mice at the age of 9-month were tested on Y maze, measuring one kind of spatial working and reference memory based on the behaviors on the spontaneous alternations. PS19 mice (Tau mice) exhibited impairment on the Y-maze test, when compared to wild type (WT), transgenic mice expressing only transactivator ((TA) under the neuronal CamkIIa promoter (Camk), or Tg-CX3CL1-ct mice. Impaired spontaneous alternation was reverted back when CX3CL1-ct was expressed in PS19 mice in Tg-CX3CL1-ct/tTA/PS19 mice (each ot represents one mouse, **. P<0.01, ***P<0.001).

FIG. 21: Expression of CX3CL1-ct in PS19 increase survival. Both male and female PS19 mice would die around 12 month of age. Mice expressing Cx3CL1-ct (Tg-CX3CL1-ct/tTA/PS19 mice) clearly increased the survival. Some of them survived beyond 20 months of age.

### DETAILED DESCRIPTION

CX3CL1, also known as fractalkine, is best known for signaling activity through interactions with its cognate receptor CX3CR1. As described herein, the intracellular domain of CX3CL1 (CX3CL1-ICD), generated upon sequential cleavages by α-/β-secretase and γ-secretase, has a back-signaling activity. A synthetic peptide derived from CX3CL1-ICD, named Tet34, was fused with a 13-amino acid tetanus sequence at the N-terminus to facilitate translocation into neuronal cells. Treatment of mouse neuroblastoma Neuro-2A cells with Tet34, but not its scrambled control (Tet34s), induced cell proliferation, manifested by changes in protein levels including Foxo-1,-3, cyclin D1, PCNA, Sox5 and cdk2. Further biochemical assays reveal elevation of phosphorylated insulin receptor β subunit, insulin-like growth factor-1 (IGF-1) receptor β subunit and insulin receptor substrates as well as activation of Akt. Transgenic mice overexpressing membrane-anchored C-terminal CX3CL1 (CX3CL1-ct) also exhibited activation of insulin/IGF-1 receptor signaling. Remarkably, this Tet34 peptide, but not Tet34s, would protect endoplasmic reticulum from stress and cellular apoptosis when Neuro-2A cells were challenged with toxic oligomers of β-amyloid peptide or hydrogen peroxide. These results suggest CX3CL1-ICD has a translational potential for neuroprotection in Alzheimer's disease and for disorders resulting from insulin resistance.

Recently, an intrinsic back-signaling activity of CX3CL1 was identified that results from its intracellular domain (CX3CL1-ICD), which is generated after sequential cleavage of membrane-bound CX3CL1 by α, β, and γ-secretases. Like the Notch intracellular domain (NICD), CX3CL1-ICD can also translocate into the cell nucleus to alter expression of many genes. The intracellular domain of CX3CL1 regulates adult neurogenesis and Alzheimer's amyloid pathology. This signaling event is independent of CX3CL1-CX3CR1 interactions and has its own signaling properties. When transgenic mice overexpress the C-terminus of CX3CL1 in neurons (Tg-CX3CL1-ct mice), enhanced neurogenesis in both the subventricular and subgranular zones is observed. Importantly, this enhanced neurogenesis mitigates neuronal loss in Alzheimer's disease (AD) mice such as 5xFAD mice, which exhibit neurodegeneration due to excessive amyloid deposition resulting from overexpressed mutant amyloid precursor protein and presenilin-1, and PS19 mice, which overexpress mutant tau protein and show broad neuronal loss.

Disclosed herein is a synthetic peptide, named Tet34, which was tagged with a 13-amino acid tetanus sequence at the N-terminus to facilitate its specific binding to GT1b ganglioside receptors, which are expressed on the surface of neuronal cells. This Tet34 peptide was effectively uptaken by neuroblastoma neuro-2a (N2A) cells and primary hippocampal neurons, entered the nucleus in a time-dependent manner. Remarkably, N2A cells treated with Tet34 showed activation of not only the TGFβ signaling pathway as described previously, but also the insulin receptor β (InsRβ) and insulin-like growth factor receptor 1β (IGFR1β) signaling pathways.

In addition, downstream signaling molecules in the insulin/IGF-1 pathways were examined, and Tet34 significantly elevated phosphorylation of Foxo-3. Foxo proteins, a subgroup of the Forkhead family of transcription factors characterized by a conserved forkhead helix loop DNA binding domain (FOX), have diverse cellular functions and are known to have roles in stress, aging, apoptosis and cell-cycle regulation. Suppression of apoptotic marker proteins as well as upregulation of multiple cellular proliferation markers, consistent with the observed cell proliferation in N2A cells treated with Tet34. The activation of insulin/IGF-1/Foxo signaling was further validated in Tg-CX3CL1-ct/tTA mice, in which transgene was induced in the early adult stage. Thus, the synthetic peptide retains the inherent signaling induction properties of CX3CL1-ICD. This small peptide can be used in therapeutic applications to counteract neuronal loss in Alzheimer's disease and other neurodegenerative diseases.

In an aspect, a polypeptide comprises an amino acid sequence at least 70%, 75%, 80%, 85%, 90%, 91%, 92%, 93%, 94%, 95%, 96%, 97%, 98%, 99%, or 100% identical to RKMAGEMAEGLRYIPRSCGSNSYVLVPV (SEQ ID NO:1).

In an aspect, a variant of SEQ ID NO: 1 is a “conservatively modified variant”, where the alteration results in the substitution of an amino acid with a chemically similar amino acid. Conservative substitution tables providing functionally similar amino acids are well known in the art.

In an aspect, the peptide is a modulator of an insulin or insulin growth factor-1 (IGF-1) as determined by Western Blot.

In certain embodiments, the polypeptide comprises RKMAGEMAEGLRYIPRSCGSNSYVLVPV (SEQ ID NO: 1).

As used herein, a polypeptide may include one or more modified or artificial amino acids, such as D-amino acids, as well as modifications such as glycosylation or PEGylation.

In an aspect, the polypeptide further comprises a heterologous peptide, a detectable label, or both.

As used herein, a heterologous peptide is a polypeptide sequence (e.g., a fusion partner) that is not naturally found with SEQ ID NO: 1. The fusion of one polypeptide (or its coding sequence) with a heterologous polypeptide (or polynucleotide sequence) does not result in a polypeptide or polynucleotide sequence that can be found in nature. Exemplary heterologous peptides include a poly-His tag, or a poly-Arg tag, or a signal peptide.

In certain embodiments, the polypeptide further comprises a heterologous peptide tag. In certain embodiments, the heterologous peptide tag is a signal peptide such as a neuron targeting tag. In certain embodiments, the polypeptide further comprises the neuron targeting tag is at least 70%, 75%, 80%, 85%, 90%, 91%, 92%, 93%, 94%, 95%, 96%, 97%, 98%, 99%, or 100% identical to HLNILSTLWKYRC (SEQ ID NO: 2). In certain embodiments, the 13-amino acid tetanus sequence fused at the N-terminus is HLNILSTLWKYRC.

Additional signal peptides include the signal peptides from tissue plasminogen activator, insulin, and neuron growth factor, and juvenile hormone esterase of Heliothis virescens.

In an aspect, a “detectable label,” or “detectable moiety” is a composition detectable by radiological, spectroscopic, photochemical, biochemical, immunochemical, chemical, or other physical means. For example, useful labels include radioisotopes such as ³²P, fluorescent dyes, electron-dense reagents, enzymes (e.g., as commonly used in an ELISA), biotin, digoxigenin, or haptens and proteins that can be made detectable, e.g., by incorporating a radioactive component into a polypeptide or used to detect antibodies specifically reactive with the polypeptide. Typically a detectable label is a heterologous moiety attached to a probe or a molecule (e.g., a protein or nucleic acid) with defined binding characteristics (e.g., a polypeptide with a known binding specificity or a polynucleotide), so as to allow the presence of the probe/molecule (and therefore its binding target) to be readily detectable. The heterologous nature of the label ensures that it has an origin different from that of the probe or molecule that it labels, such that the probe/molecule attached with the detectable label does not constitute a naturally occurring composition (e.g., a naturally occurring polynucleotide or polypeptide sequence).

In certain embodiments, the polypeptide is a synthetic polypeptide or an isolated polypeptide. In certain embodiments, the polypeptide is a synthetic polypeptide. In certain embodiments, the polypeptide is an isolated polypeptide. The term “isolated” nucleic acid or polypeptide molecule means a nucleic acid or polypeptide molecule that is separated from other nucleic acid or polypeptide molecules that are usually associated with the isolated nucleic acid or polypeptide molecule.

Also included are nucleic acids expressing the polypeptides described herein. Unless otherwise indicated, a particular nucleic acid sequence also implicitly encompasses conservatively modified variants thereof (e.g., degenerate codon substitutions). With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

Also included are expression cassettes comprising a nucleic acid expressing the polypeptides described herein. An “expression cassette” is a nucleic acid construct, generated recombinantly or synthetically, with a series of specified polynucleotide elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter.

A “promoter” is defined as an array of nucleic acid control sequences that direct transcription of a polynucleotide sequence. As used herein, a promoter includes necessary polynucleotide sequences near the start site of transcription, such as, in the case of a polymerase II type promoter, a TATA clement. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a polynucleotide expression control sequence (such as a promoter, or array of transcription factor binding sites) and a second polynucleotide sequence, wherein the expression control sequence directs transcription of the polynucleotide sequence corresponding to the second sequence.

Also included is a vector, such as a cloning vector or expression vector, comprising the expression cassette. Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 carly promoter, SV40 later promoter, metallothionein promoter, murine mammary tumor virus promoter, Rous sarcoma virus promoter, polyhedrin promoter, or other promoters shown effective for expression in eukaryotic cells.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

In an aspect, disclosed is a pharmaceutical composition comprising at least one of the peptides disclosed herein and at least one pharmaceutically acceptable excipient.

The polypeptides described herein may be in the form of an acid or base addition salt.

The disclosed pharmaceutical compositions may be prepared in various forms, such as capsules, suppositories, tablets, food/drink and the like. Optionally, the disclosed pharmaceutical compositions may include various pharmaceutically acceptable excipients, such as microcrystalline cellulose, mannitol, glucose, defatted milk powder, polyvinylpyrrolidone, starch, or a combination thereof.

The polypeptides as described herein may, in accordance with the disclosure, be administered in single or divided doses by the oral, parenteral or topical routes. Administration of the polypeptides may range from continuous (intravenous drip) to several administrations per day (for example, Q.I.D.) and may include administration routes such as oral, topical, parenteral, intramuscular, intravenous, sub-cutaneous, transdermal (which may include a penetration enhancement agent), buccal, sublingual, intranasal, intraocular, intrathecal, vaginal, and suppository administration, among other routes of administration. The term “parenteral” as used herein includes subcutaneous, intravenous, intramuscular, intra-articular, intra-synovial, intrasternal, intrathecal, intrahepatic, intralesional and intracranial injection or infusion techniques. Enteric coated oral tablets may be used to enhance bioavailability of the compounds from an oral route of administration. The most effective dosage form will depend upon the pharmacokinetics of the polypeptides as well as the type, location and severity of disease, condition or symptom, and the health of the patient. Administration of polypeptides as sprays, mists, or aerosols for intra-nasal, intra-tracheal or pulmonary administration may also be used. The present disclosure therefore also is directed to pharmaceutical compositions comprising an effective amount of polypeptides as described herein or a pharmaceutically acceptable salt thereof, optionally in combination with a pharmaceutically acceptable carrier, additive or excipient. Polypeptides of the present disclosure may be administered in immediate release or sustained or controlled release forms. Sustained or controlled release forms are preferably administered orally, but also in suppository and transdermal or other topical forms. Intramuscular injections in liposomal form or in depot formulation may also be used to control or sustain the release of compound at an injection site.

The polypeptides as described herein may be formulated in a conventional manner using one or more pharmaceutically acceptable carriers and may also be administered in controlled-release formulations. It should also be understood that a specific dosage and treatment regimen for any particular patient will depend on the judgment of the treating physician as based upon a variety of factors, including the activity and bioavailability of the specific polypeptides employed, the age, body weight, general health, sex, diet, time of administration, rate of excretion, drug (therapeutic agent) combination, and the severity of the particular disease or condition being treated.

A patient or subject in need of therapy using a compound according to the methods described herein can be treated by administering to the patient (subject) an effective amount of the polypeptides, for example a pharmaceutical composition including the polypeptides, according to the present disclosure, either alone, or in combination with another known therapeutic agent.

In certain aspects, the polypeptide is conveniently administered in any suitable unit dosage form, including but not limited to a dosage form containing less than 1 milligrams (mg), 1 mg to 3000 mg, or 5 mg to 500 mg of active ingredient per unit dosage form. An oral dosage of about 25 mg-250 mg is often convenient.

In certain aspects, the polypeptides is preferably administered to achieve peak plasma concentrations of the active compound of about 0.00001-30 millimole (mM), preferably about 0.1-30 micromole (μM). This may be achieved, for example, by the intravenous injection of a solution or formulation of the polypeptides, optionally in saline, or an aqueous medium or administered as a bolus of the polypeptides. Oral administration may also be appropriate to generate effective plasma concentrations of polypeptides.

The concentration of polypeptides in the pharmaceutical composition will depend on absorption, distribution, metabolism, and excretion rates as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the physician administering or supervising the administration of the pharmaceutical compositions, and that the concentration ranges set forth herein are exemplary only and are not intended to limit the scope or practice of the claimed pharmaceutical composition. The polypeptides may be administered at once, or may be divided into a number of smaller doses to be administered at varying intervals of time.

In an aspect, disclosed is a method for preventing or treating a neurodegenerative disease in a subject, the method comprising: providing and administering to the subject a polypeptide disclosed herein, and/or the pharmaceutical composition disclosed herein. In an aspect, the method comprises treating a subject exhibiting a symptom (which includes one or more symptoms) of a neurodegenerative disease and/or diagnosed with a neurodegenerative disease.

In certain embodiments, the neurodegenerative disease is Alzheimer's disease, Parkinson's disease, amyotrophic lateral sclerosis, ataxia such as Friedreich ataxia, Huntington's disease, Lewy body disease, spinal muscular atrophy, multiple system atrophy, motor neuron disease, progressive supranuclear palsy (PSP), corticobasal syndrome (CBS), frontal dementia diseases, or a combination thereof. In certain embodiments, the neurodegenerative disease is Alzheimer's disease, Parkinson's disease, or a combination thereof. In certain embodiments, the neurodegenerative disease is Alzheimer's disease. In certain embodiments, the neurodegenerative disease is Parkinson's disease. In certain embodiments, the method is administered as a monotherapy or as a part of combination therapy. In certain embodiments, the subject is human.

Exemplary symptoms of neurodegenerative disease include confusion, disinhibition, apathy, anxiety, memory loss, difficulty thinking or concentrating, behavior changes, mood changes, depression, delusions, hallucinations, tingling or numbness, pain, muscle spasms, weakness, paralysis, coordination issues, fatigue, slowed movements, shaking, tremors, balance problems, shuffling steps, hunched posture, loss of muscle control, weakness, paralysis, and combinations thereof.

In an aspect, the polypeptide may be co-administered with an agent used for treatment of a neurodegenerative disease such as Huntington's disease, amyotrophic lateral sclerosis (ALS), Parkinson's disease, and Alzheimer's disease.

In an aspect, disclosed is a method for preventing or treating diabetes in a subject, the method comprising administering to the subject a polypeptide disclosed herein, and/or the pharmaceutical composition disclosed herein. In an aspect, the method comprises treating a subject exhibiting a symptom (which includes one or more symptoms) of diabetes and/or diagnosed with diabetes, comprising administering to the subject a polypeptide disclosed herein, and/or the pharmaceutical composition disclosed herein.

In an aspect, diabetes includes Type II diabetes, due to the impaired insulin receptor function in many diabetic patients.

In an aspect, a symptom of Type II diabetes include frequent urination, excessive thirst, fatigue, unexpected weight loss, thrush, slow healing, blurred vision, increased hunger, and a combination thereof.

In an aspect, the polypeptide may be co-administered with an agent used for treatment of diabetes, including conditions as diabetic neuropathy, nephropathy, and retinopathy.

In an aspect, disclosed is a method for preventing or treating aging in a subject, the method comprising administering to the subject a polypeptide disclosed herein, and/or the pharmaceutical composition disclosed herein. Most neurodegenerative diseases and diabetes are highly influenced by aging, in that the levels of insulin-like growth factor-1 (IGF-1) decrease with age, and CX3CL1-ICD elevates IGF1 levels. In this sense, it may have anti-aging effect. In an aspect, the method comprises treating a subject exhibiting a symptom (which includes one or more symptoms) of aging, comprising administering to the subject a polypeptide disclosed herein, and/or the pharmaceutical composition disclosed herein.

In an aspect, an aging symptom includes exacerbation of a neurogenerative disease and/or diabetes symptom as described above.

In an aspect, the polypeptide may be co-administered with an agent used for treatment of a neurogenerative disease and/or diabetes as described above.

The invention is illustrated by the following non-limiting examples.

### EXAMPLES

### Example 1: CX3CL1 Intracellular Peptide Translocates to the Cell Nucleus and Induces Cell Proliferation

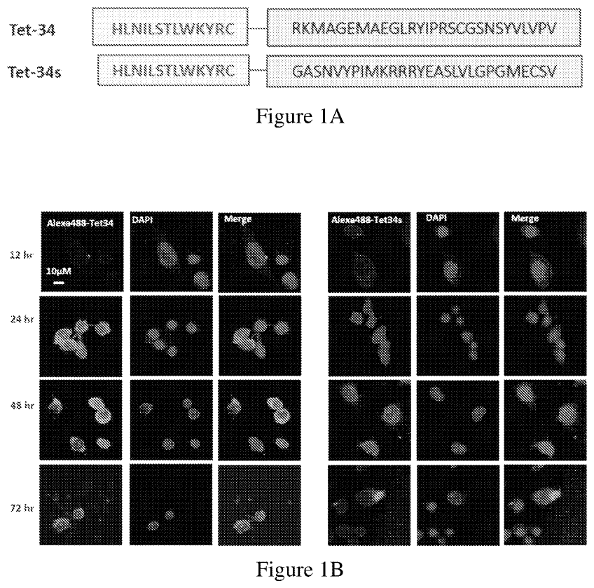

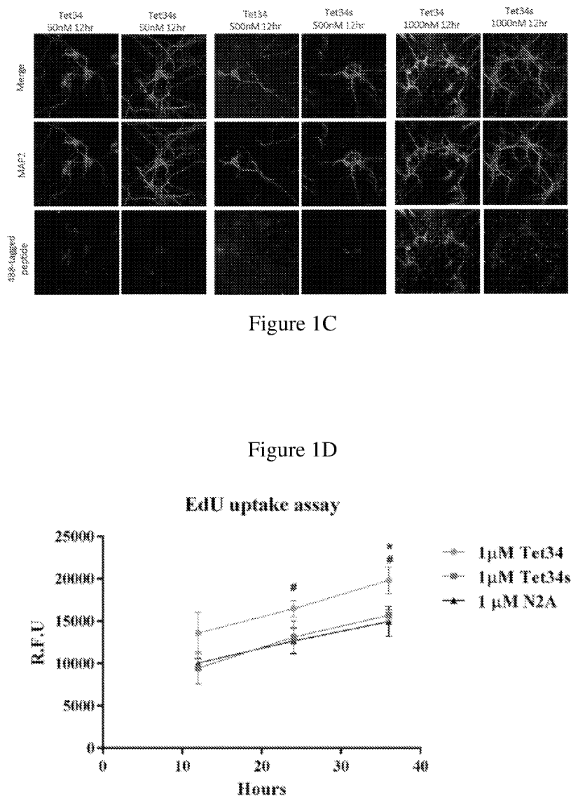

A synthetic peptide was designed from the sequence of CX3CL1 intracellular domain (CX3CL1-ICD) and fused with a 13-amino acid Tet sequence derived from tetanus toxin at the N-terminus to improve its specific uptake by neurons and neural stem cells (FIG. 1A, illustration of peptide sequences). This peptide, named as Tet34, and the control peptide Tet34s which has the scramble order of sequence within the CX3CL1-ICD region, were tested for their ability to penetrate into neuronal cells. Since CX3CL1 C-terminal antibody would only recognize Tet34 but not Tet34s, a batch of these peptides were also tagged with Alexa 488 (Alexa-Fluor® 488-Tet34 or Alexa-Fluor® 488-Tet34s) for monitoring neuronal cell uptake. We showed that Alexa-Fluor® 488-Tet34 was more effective in penetration into mouse neuroblastomas Neuro-2a (N2A) cells than Alexa-Fluor® 488-Tet34s at concentrations of 125 nM to 200 nM (FIG. 1B). During the monition between 12 to 72 hrs, clear uptake of Alexa-Fluor® 488-Tet34 was seen at 24 hrs and peaked around 48 hrs.

We also examined uptake of Alexa-Fluor® 488-Tet34 in primary neurons cultured from E16.5 mouse hippocampus at varying concentrations and time points. Alexa-Fluor® 488-Tet34 and Alexa-Fluor® 488-Tet34s were added at concentrations of 50, 500 and 1000 nM at 7 DIV to primary neuron cultures. After treatment for 12- and 24-hours, cells were fixed and stained with MAP2. Alexa-Fluor® 488-Tet34 signal was robustly detected in neuronal cell bodies after 12-24 hours at a concentration of 1000 nM (FIG. 1C). At the same concentration, Alexa-Fluor® 488-Tet34s was not easily detected, suggesting that the sequence of CX3CL1-ICD facilities the uptake of Tet34, although slightly different uptake kinetics between primary hippocampal neurons and N2A cells.

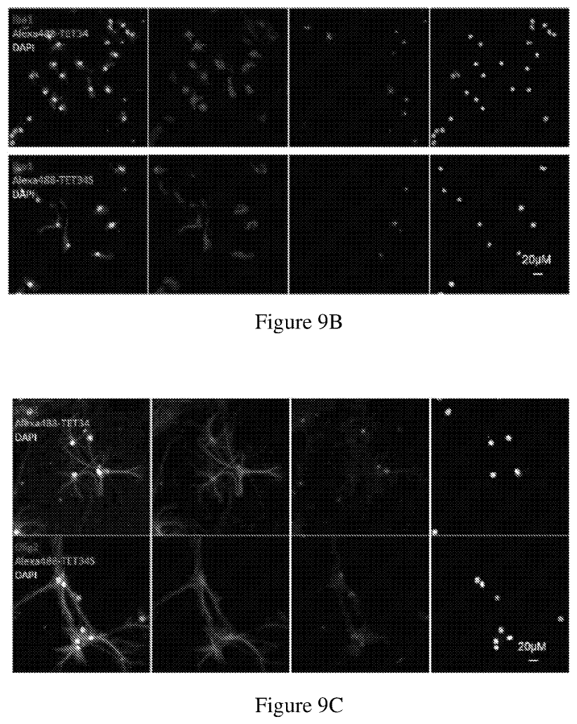

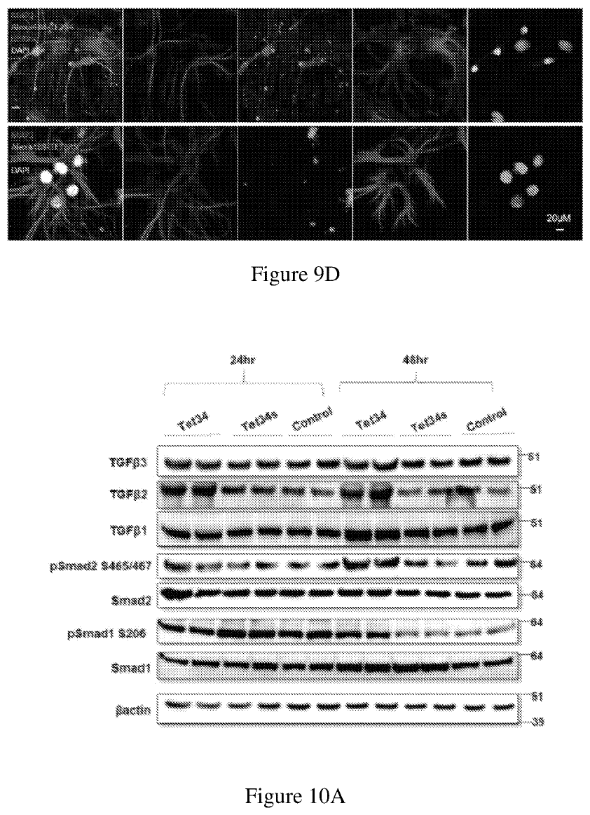

To determine if Alexa-Fluor® 488-Tet34 uptake was specific to neurons, we waited until 12 DIV without treatment of AraC to allow proliferation of glial cells. After treatment for 12 hours, we began to observe uptake of Alexa-Fluor® 488-Tet34 by MAP2-marked neuron, but much less by astrocytes, labeled by GPAP antibody or Iba1-labeled microglia or Olig1-labeled oligodendrocytes (FIGS. 9A-D). Again, uptake of Alexa-Fluor® 488-Tet34s by neurons or astrocytes was weak. We could not exclude the possibility that a fraction of Alexa-Fluor® 488-peptides were phagocytosed and degraded by glial cells. Based on our observations, we conclude that peptide Alexa-Fluor® 488-Tet34 is primarily up-taken by neurons. The CX3CL1-ICD sequence appears to facilitate cellular and nuclear uptake when comparing the neuronal uptake by Alexa-Fluor® 488-Tet34 and Alexa-Fluor® 488-Tet34s.

During the cellular assay, we noted that N2A cells, when treated with Alexa-Fluor® 488-Tet34, grew more densely. Therefore, N2A cells were treated with non-fluorescent Tet34 peptides at the concentrations of 1 μm for 12, 24 and 36 hrs to monitor cell proliferation by EdU incorporation assay. Cells treated with Tet34 had significantly increased EdU incorporation at 24 hrs and 36 hrs compared to untreated controls or Tet34s treated cells (FIG. 1D), indicating that Tet34 likely has a role in upregulation of cellular proliferation.

### Example 2: Peptide Tet34 Activates the Insulin/IGF1 Signaling Pathways in Cultured Cells

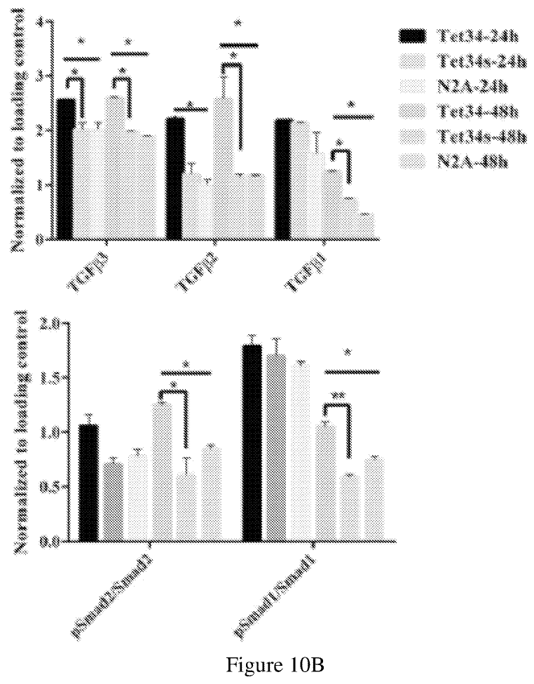

To determine how Tet34 upregulate cell proliferation, we first examined whether this fusion Tet34 peptide retains the activity of inducing TGFβ signaling, similar to the expression of CX3CL1-ct in mice. We found that treatment of N2A cells with 2 μM of Tet34 for 24 hrs and 48 hrs significantly increased TGFβ2 and TGFβ3 expression, while TGFβ1 levels were elevated only at 48 hrs, as compared to Tet34s treatment (FIGS. 10A-B), indicating that Tet34 has a desired signaling activity. Elevated TGFβ2 and TGFβ3 expression induced a significant increase in phosphorylated Smad1 and Smad2 levels at 48 hrs, but not total Smad expression levels (FIGS. 10A-B). Thus, we demonstrated that fusion of Tet sequence to CX3CL1-ICD retains the activation of the TGFB/Smad signaling pathway.

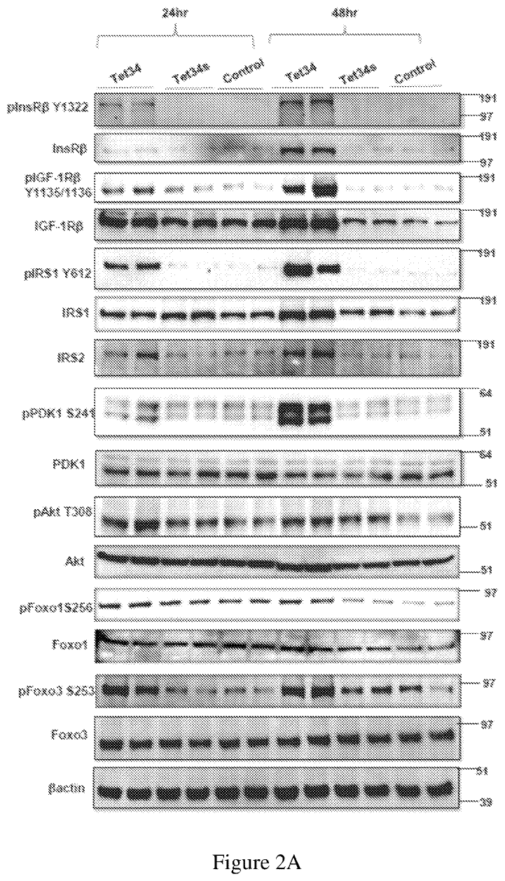

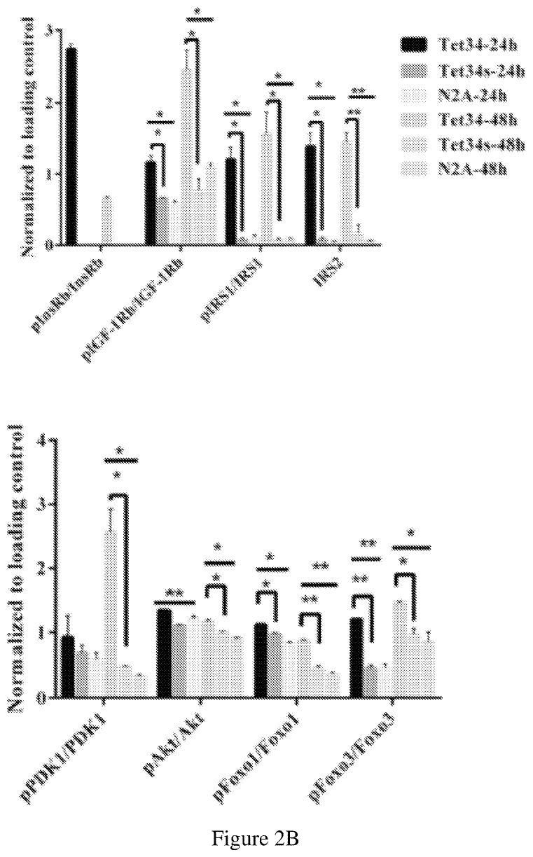

Our further biochemical exploration revealed that Tet34 treatment of N2A cells significantly upregulated expression of insulin receptor β subunit (InsRβ), which is phosphorylated when activated by insulin, when compared to Tes34s or mock treatment (FIG. 2A). A corresponding increase in the level of phosphorylated of InsRβ (pInsRβ) was more evident. This increase of pInsRβ likely induced activation of its downstream molecules insulin receptor substrate-1 and-2 (IRS1 and IRS2; FIG. 2A); the increase was visibly more at the 48 hr-treatment. Phosphorylated IRS1 was significantly increased, and confirmed by quantification (FIG. 2B). We also noted an increase in the levels of insulin growth factor 1 receptor (IGF1-R), detected by the antibody specific to the β-subunit (IGF-1Rβ). Levels of pIGF-1Rβ were notably increased as well (FIGS. 2A-2B).

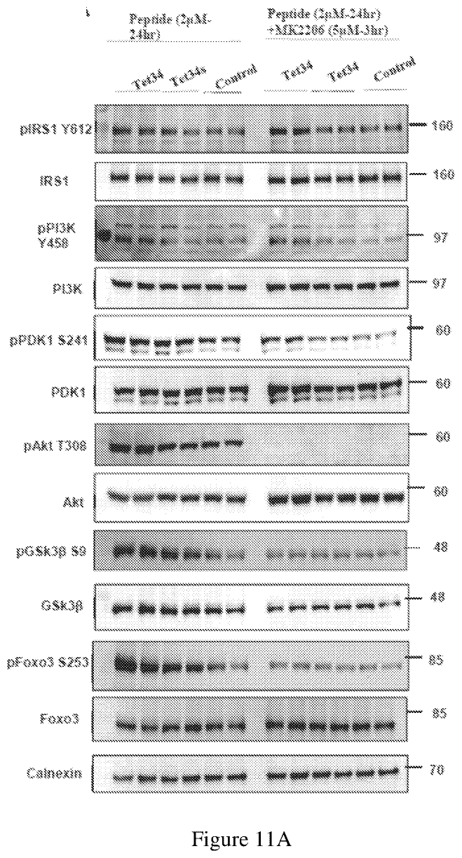

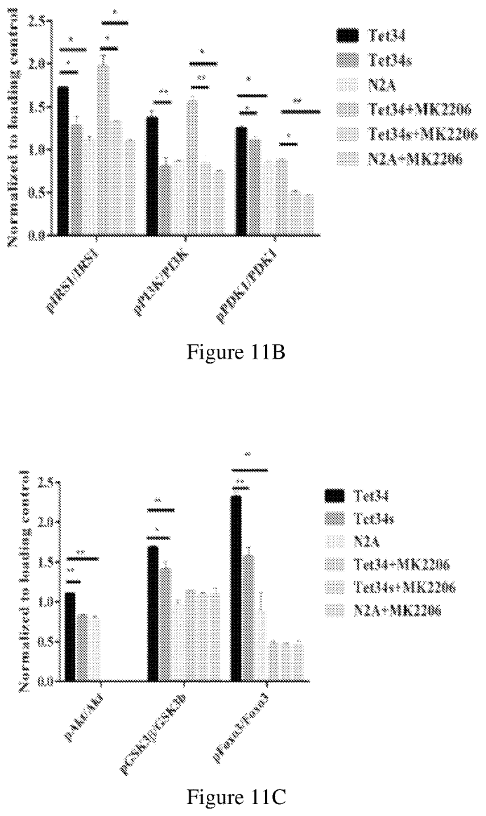

We then further examined downstream molecules and detected activation of PDK1 and Akt, as demonstrated by an increase in their phosphorylated levels but not total PDK1 or Akt (FIGS. 11A-C). Akt phosphorylation is known to be one of the key regulators of Forkhead transcription factors (Foxos). The activities of Foxos are dependent upon their subcellular localization and phosphorylation status. Nuclear Foxos actively bind to their transcriptional targets, whereas phosphorylated Foxos are shuttled out into cytoplasm, where they undergo either dephosphorylation or degradation. Tet34 treatment led to a significant increase in phosphorylation of Foxo3 with no changes in total Foxo3 expression (FIGS. 11A-C). Phosphorylation of Foxo1 was moderate but significantly increased as well, indicating downregulation of Foxo1 and Foxo3 activity.

We also repeated the same set of experiment by including pharmacological inhibition of Akt activation with compound MK2206, in order to probe the effect of Akt activation on Foxo activity in N2A cells, which were treated with 2 μM of Tet34 or controls for 24 hrs. It appeared that inhibition of Akt phosphorylation would diminish phosphorylation of GSK3β and Foxo3 (FIGS. 11A-C), indicating a critical role of Akt activity in the Akt/Foxo pathway.

### Example 3: CX3CL1-ICD Enhances Insulin/IGF-1 Signaling Pathways in Vivo

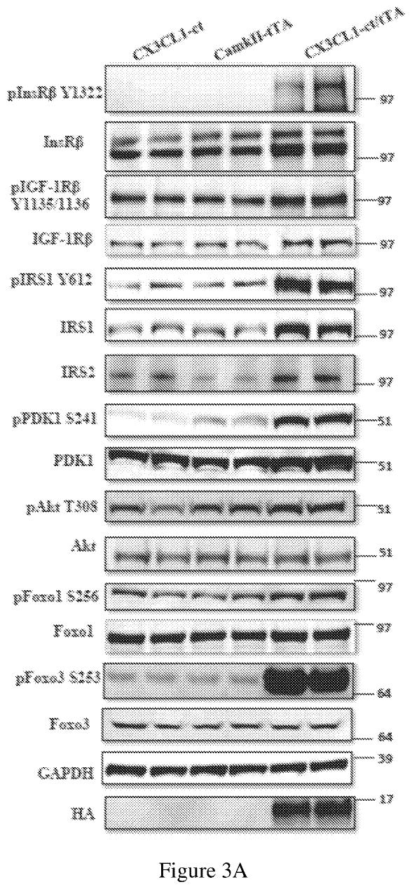

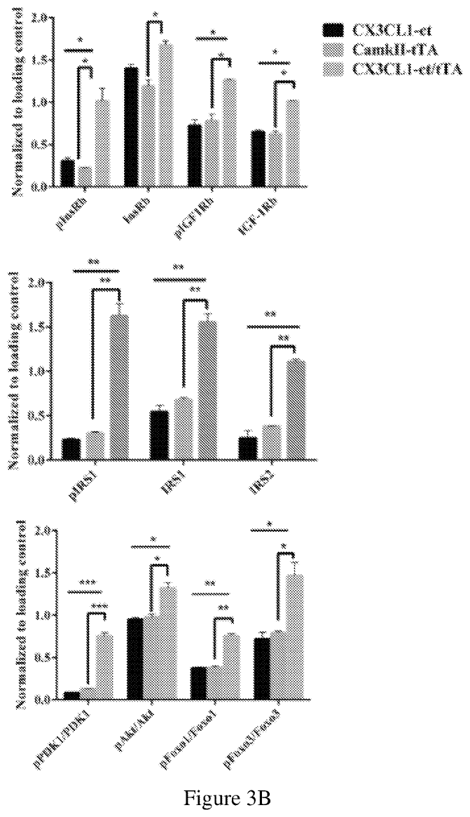

Transgenic mice overexpressing membrane-anchored C-terminal CX3CL1 fragment (Tg-CX3CL1-ct) were generated by utilizing the tetracycline-inducible promoter as described previously. This previous study in this model focused on developmental neurogenesis; increased neuron numbers in hippocampal and cortical regions have been demonstrated. To determine whether expression of CX3CL1-ct transgene in adult Tg-CX3CL1-ct/tTA mice would have insulin/IGF-1 signaling functions, we first treated Tg-CX3CL1-ct/tTA mice and their control litters with doxycycline in the drinking water, starting from the mating stage to suppress transgene expression, and doxycycline was removed at the age of postnatal day 45 (P45) to turn on CX3CL1-ct expression. After one month of induced expression, mice were sacrificed, and protein lysates were prepared from hippocampi for Western blot examination. Since CX3CL1-ct has an HA-tag on its C-terminus, HA expression was observed only in Tg-CX3CL1-ct/tTA mice. When compared to non-transgene-expressing littermates (WT/CX3CL1-ct, CamKII-tTA mice), we found that induced expression of CX3CL1-ct elevated protein levels of both IGF-1Rβ and InsRβ signaling molecules (FIG. 3A). Increased phosphorylation of insulin receptor was the most prominent while the increase of pIGF-1Rβ was also visible (FIG. 3A). These increases were further confirmed by quantification (FIG. 3B).

Their downstream signaling molecules were also examined by the Western blot analysis. As shown in FIG. 3A, total IRS1 and IRS2 levels were visibly elevated, as was phosphorylated IRS1 levels. While total PDK1 levels were slightly elevated, phosphorylated PDK1 (pPDK1) increased significantly (FIG. 3A), indicating a strong activation of PDK1 (quantified in FIG. 3B). While total Akt levels were not changed, elevation in Akt phosphorylation was significant. Activity of the transcription factors Foxo1 and Foxo3 were both repressed, as the levels of both phosphorylated Foxo1 (pFoxo1) and Foxo3 (pFoxo3) were significantly increased with no changes in total Foxo1 and Foxo3 levels (FIGS. 3A-3B).

Together, these results support that CX3CL1-ICD has a role in the activation of both IGF-1Rβ and InsRβ signaling. Insulin/IGF signaling in brains, specifically in hippocampus, has been shown to regulate spatial learning and memory, and this activation may partially explain the improved cognitive functions seen in AD mice (PS19 mice) upon overexpressing CX3CL1-ICD.

### Example 4: CX3CL1 Intracellular Domain Upregulates Cellular Proliferative Markers

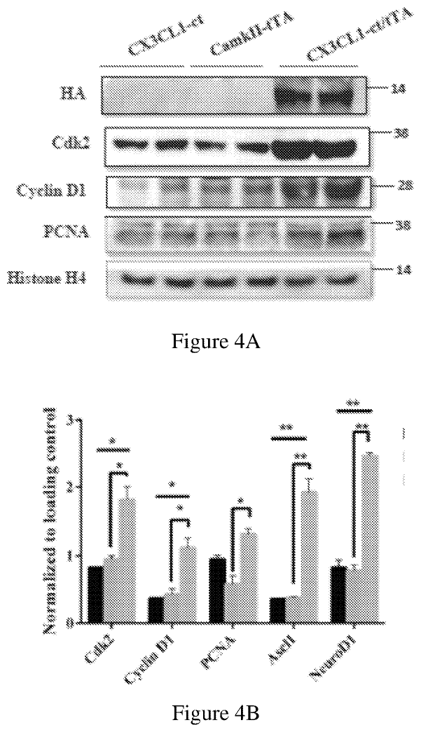

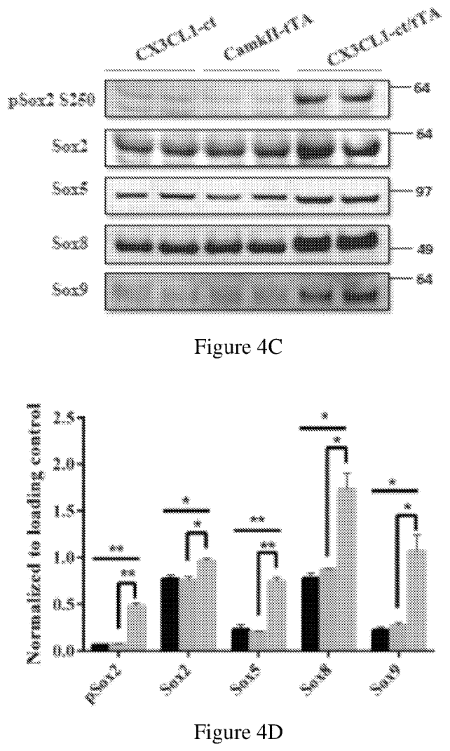

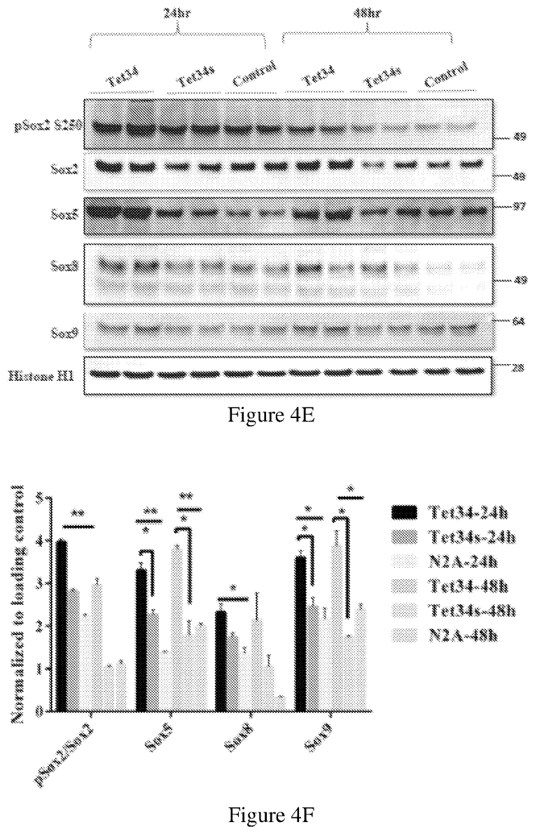

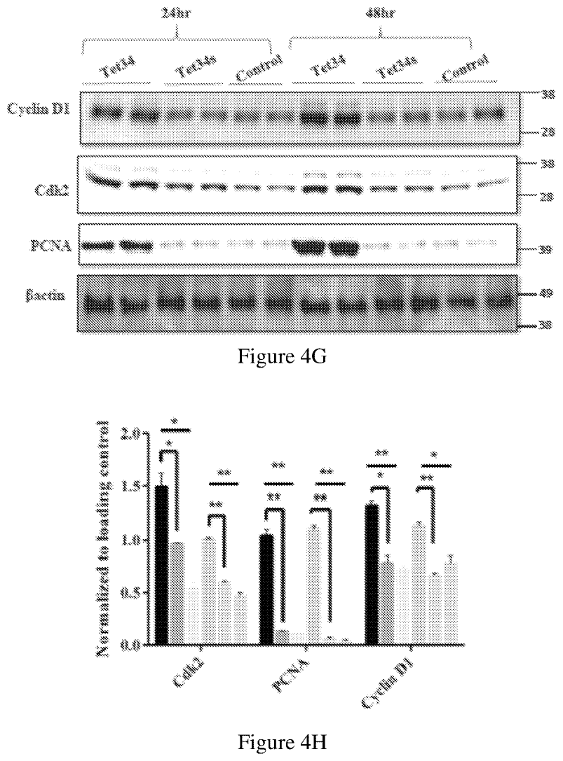

Foxo proteins are known to regulate cellular proliferation, we examined protein levels of transcription factors that control neuronal proliferation in Tg-CX3CL1-ct/tTA mice. We found that proteins important for cell-cycle progression such as Cyclin D1, Cdk2, and PCNA were significantly elevated (FIGS. 4A-4B). This corroborates our previous findings of Tg-CX3CL1-ct/tTA mice exhibiting enhanced neuronal proliferation and maturation in the subgranular zone of the dentate gyrus.

Our previous bulk RNA sequencing results showed elevation of Sox2 and Sox5, and we indeed observed elevation of these two protein levels including phosphorylated Sox2 in the above lysates (FIG. 4C). Sox5 is a protein known to control cell cycle progression while Sox2 activation is known to play a critical role in the maintenance and differentiation of neural stem cells. Sox9 expression was significantly increased while expression of Sox8 was moderately elevated (FIGS. 4C-4D); Sox9 is known to promote both basal progenitor proliferation and gliogenesis in developing neocortex.

In our cellular assays, we showed that the levels of Sox2 and phosphorylated Sox2, Sox5, Sox8, and Sox9 were elevated even after 24-hr treatment in N2A cells (FIGS. 4E-4F). Unlike results in Tg-CX3CL1-ct/tTA mice, elevation of PCNA levels was the most obvious while cdk2, Cyclin D1 levels were significantly increased. Together, these results show that Tet34 treatment has a potent signaling activity, which promotes both cellular proliferation, in addition to neural differentiation.

### Example 5: Overexpression of CX3CL1-ICD Attenuates Neuronal Apoptosis in Mouse Brains

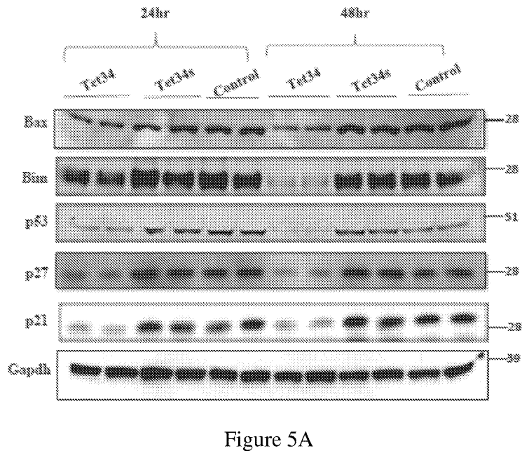

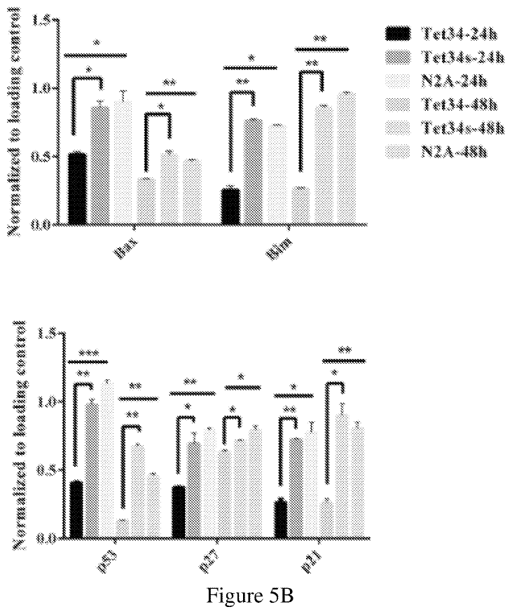

Foxos are known to be regulators of apoptotic signaling pathways in response to stress. N2A cells treated with Tet34 or Tet34s in 2% serum conditions were assayed for the expression of pro-apoptotic marker proteins such as p53, Bax and Bim; all these three genes are transcriptionally regulated by Foxos. We showed that Tet34 treatment significantly reduced expression of these apoptotic markers in comparison to scrambled Tet34s treatment or mock-treated cells (FIGS. 5A-5B). We showed that while the levels of cyclin-dependent kinase inhibitors p21 and p27 were significantly reduced (FIGS. 5A-5B), consistent with the report that p21 and p27 are transcriptionally regulated by Foxos. Significant reduction of these molecules indicates a reduction in cellular apoptosis.

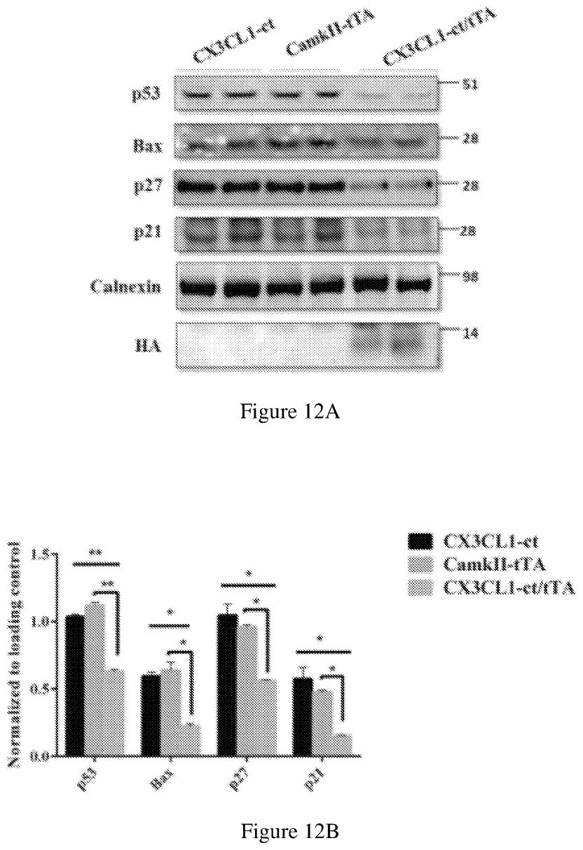

Reduction of neuronal apoptosis is intriguing. Subgranular zone (SGZ) of dentate gyrus and subventricular zone (SVZ) are active neurogenic niches in the murine brain. Majority of the newborn cells in the SGZ and SVZ undergoes apoptosis in different stages of neuronal differentiation and maturation into neurons. In line with our previous findings of enhanced neurogenesis (NeuN positive cells) in the SGZ and SVZ of Tg-CX3CL1-tTA, we found reduction of apoptotic markers in Tg-CX3CL1-tTA mouse hippocampi (FIGS. 12A-B). Hence, CX3CL1-ICD may also promote neuronal survival by attenuating apoptosis.

### Example 6: Tet34 Exerts Neuroprotection by Attenuating Aβ-Induced Cellular Stress and Toxicity

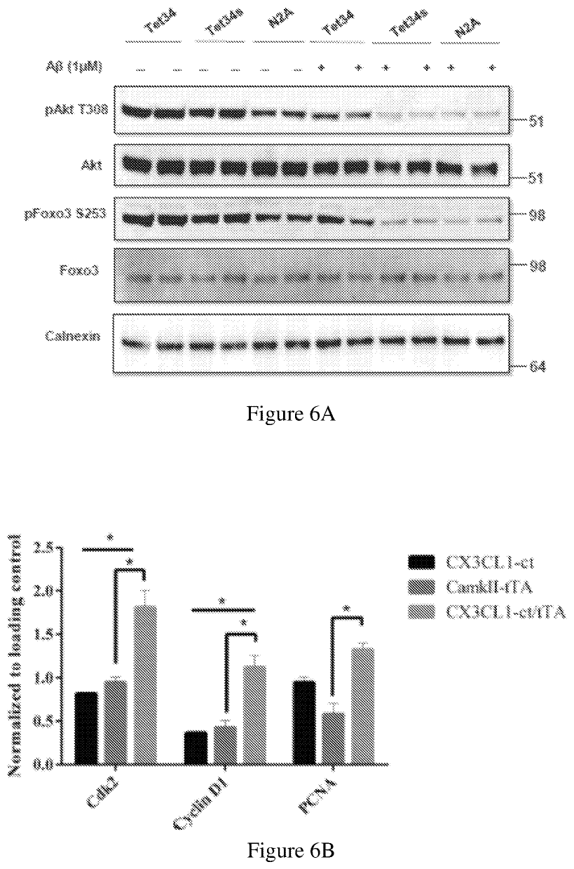

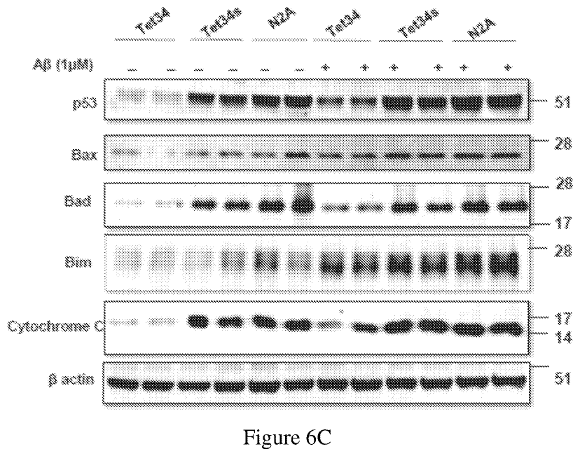

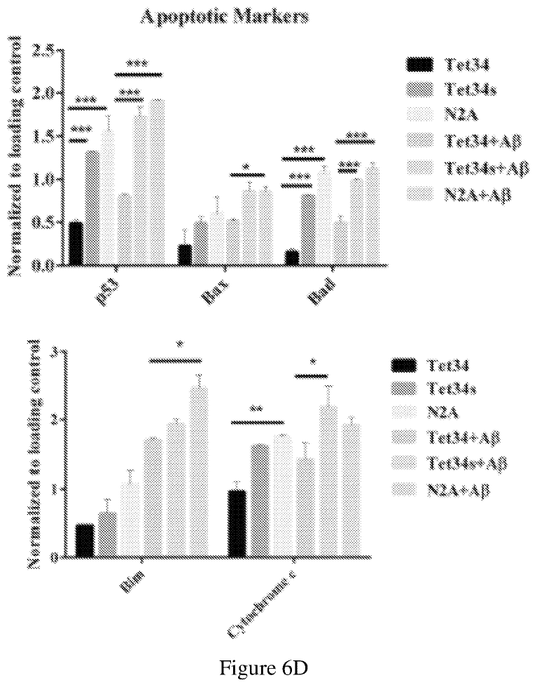

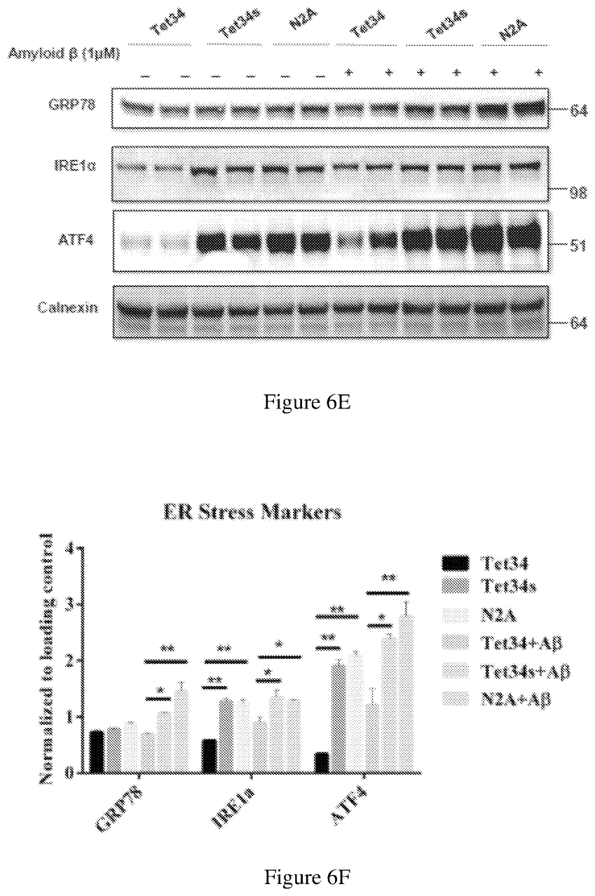

It is known that Aβ treatment of N2A cells induces cellular stress and apoptosis in a dose-dependent manner. To determine whether CX3CL1-ICD would exert a neuroprotective effect, we addressed this question in cultured N2A cells. N2A cells were treated with 2 μM of Tet34 and Tet34s for 24 hrs in 2% serum conditions prior to incubation with 1 μM of oligomeric Aβ₁₋₄₂ (briefed as Aβ thereafter) for 12 hrs. We showed that Akt protein levels, and its kinase activity, were decreased by Aβ treatment. Correspondingly, less phosphorylation of its substrate, Foxo3, was detected (FIG. 6A). N2A cells treated with Tet34 along with Aβ showed clear resilience to Aβ-mediated reduction of pAkt and pFoxo3 levels, which were confirmed by quantification (FIG. 6B).

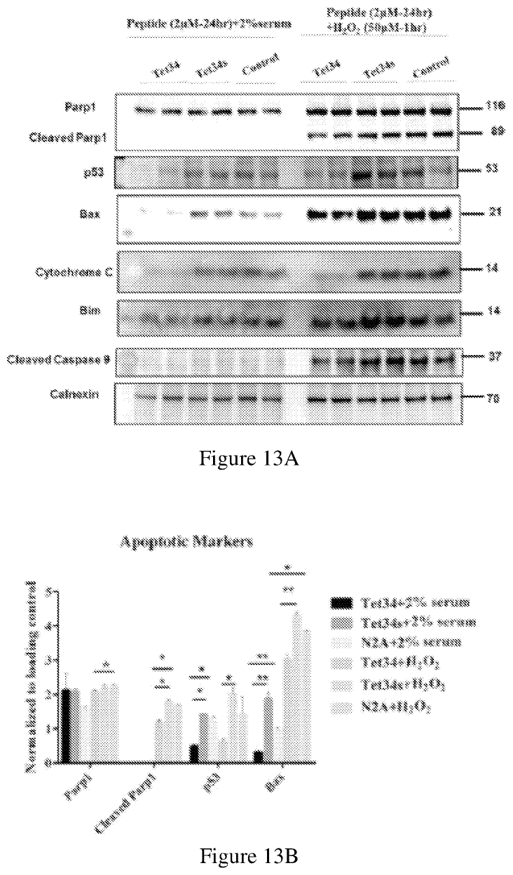

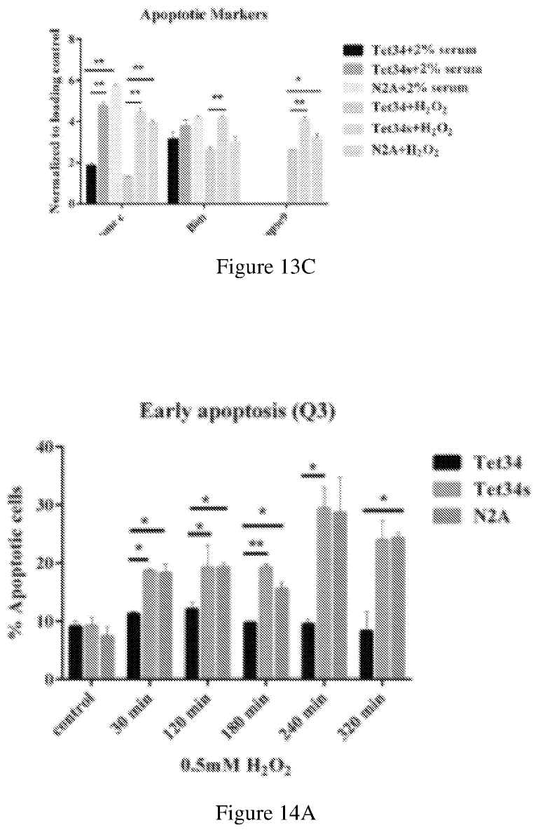

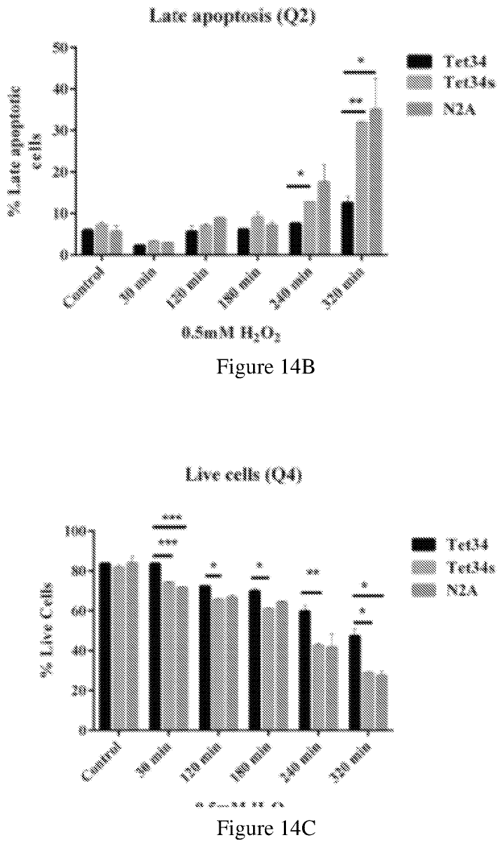

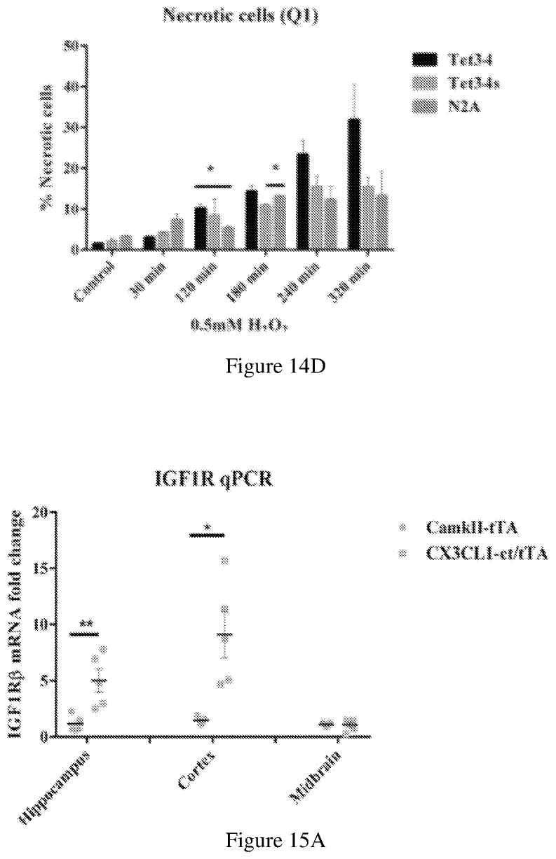

We also noted that Aβ treatment of N2A cells predominantly activated p53, Bad and Bim, which were elevated when comparing with and without Aβ treatment in N2A cells (FIGS. 6C-6D). Changes in Bax were less obvious. Tet34-treated N2A cells appeared to protect cellular apoptosis as levels of p53, Bad and Bim were significantly lowered when comparing to Tet34s or mock treated conditions with or without Aβ challenges. Relatively, Tet34 had less of an attenuating effect on Aβ-mediated Bax activation (P=0.0437) (FIGS. 6C-6D). Reduction of cytochrome C release by Tet34 treatment was substantial when comparing Tet34-treated with two control groups (FIGS. 6C-6D); Aβ induced increase in cytochrome C release and this toxic effect was clearly mitigated. These results are in line with the anti-apoptotic effects exerted by Tet34. We further confirmed the anti-apoptotic effects of Tet34 peptides on N2A cells treated with hydrogen peroxide. Tet34 attenuated the upregulation of various apoptotic markers in both 2% serum conditions as well as in hydrogen peroxide treated groups (FIGS. 13A-C).

In addition, Aβ is known to induce ER stress in neurons. Therefore, we asked whether Aβ treatment in N2A cells would also induce endoplasmic reticulum (ER) stress and be protected by Tet34 treatment. We found significant increase in the levels of 78-kDa glucose-regulated protein (GRP78), inositol-requiring enzyme 1α (IRE1α), and activating transcription factor 4 (ATF4) proteins in cells treated with Aβ (FIGS. 6E-6F). The expression of these markers was lessened by Tet34 treatment in N2A cells. The 2% serum growth condition by itself also induces low level of stress, and such a low level of stress could also be alleviated by Tet34 treatment. Thus, these results demonstrate that Tet34 exerts protective effects against Aβ-mediated cellular stress and apoptosis.

### Discussion

With the increase in median life expectancy, the onset of age-dependent neurodegenerative diseases has become more prevalent, which has resulted in one of the major social and economic burdens in modern society. Thus, finding ways to treat neurodegenerative diseases is an urgent task. In this study, we demonstrate that a short peptide, named Tet34, derived from CX3CL1-ICD, has a therapeutic potential by mitigating neuronal stress and apoptosis.

In this study, we first investigated neuronal delivery of this CX3CL1-ICD-derived short peptide by creating Tet34 peptide, and confirmed a relatively higher uptake and retention of Tet34 by N2A cells in comparison to the scrambled Tet34s (FIG. 1B). When applied in co-cultures of primary neurons and glia, accumulation of Tet34 in neurons was evident, while much less was present in glial cells (FIG. 1C FIGS. 9A-D). Consistent with our previous findings in transgenic mice expressing C-terminal Cx3CL1. Tet34 peptide induced the TGFβ/Smad signaling pathway by significantly upregulated expression of TGFβ2 and TGFβ3, as well as phosphorylation of Smad2 and Smad1 (FIGS. 10A-B), indicating that fusion of Tet sequences did not affect the effect of CX3CL1-ICD.

In cultured studies, we observed significantly increased cellular proliferation in N2A cells treated with Tet34 (FIG. 1B). We previously showed enhanced neurogenesis and significantly more mature neurons in both dentate gurus and cortical regions of Tg-CX3CL1-ct/tTA mice likely due to the activated TGFβ/Smad signaling pathway. One intriguing question is whether more mature neurons in the cortical region is due to enhanced adult neurogenesis or decreased apoptosis. It is understood that most of the (50%-70%) newborn cells in the SGZ and SVZ undergo programmed cellular death prior to maturation and integration into the neuronal network, and adult neurogenesis in SVZ is less well understood in term of migration of newborn neurons to the cortical region. With the finding of elevated levels of IGF in our bulk RNAseq experiments, we asked whether some pro-survival pathways might be playing a crucial role in the survival of the newborn neurons in transgenic mice. In this study we demonstrated that Tet34 peptides activated insulin/IGF1 signaling pathway. This is the first evidence that intracellular domain of CX3CL1 plays a role in mitigating cellular stress and apoptosis through Akt/Foxo pathways.

In both in vitro and in vivo experiments, we found that peptide Tet34, derived from CX3CL1-ICD, is capable of inducing significant elevation of phosphorylated InsRβ (Y1322) and IGF-1Rβ (Y1134/Y1136) (FIGS. 2A-B and 3A-B).

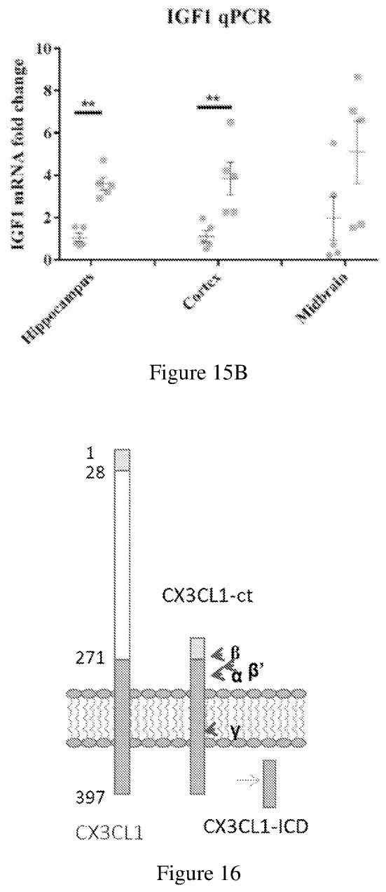

How CX3CL1-ct induces expression of insulin and insulin-like growth factor receptors is intriguing. By analyzing our bulky-seq data [Fan et al., 2019], we observed a small increase in gene expression of IGF-1R and IGF binding protein but not insulin receptors. In our RT-PCR experiments, we observed elevation of IGF and IGF-1Rβ mRNA levels (FIGS. 15A-D). While not wishing to be bound by theory, it is understood that additional posttranslational effect may also contribute to the induced expression. Insulin/IGF-1 signaling has been known to mediate cell-cycle regulation and to modulate cell survival via the downstream PDK1/Akt pathway and transcriptional molecules such as Foxos. Forkhead transcription factors (Foxos) were identified as Akt substrates, and IGF/Akt signaling regulates the activity of Foxos via their phosphorylation followed by nuclear exclusion. In our studies, the increase in Akt phosphorylation correlated with increased phosphorylation of Foxo1 and Foxo3, while changes in total Foxo levels were not evident. Phosphorylated Foxos are shuttled out from the nucleus, leading to repression of apoptotic signaling and reversion of Foxo-induced cell cycle control. Blocking Akt phosphorylation at T308 by molecule MK2206, phosphorylation of GSK3b and Foxo3 was diminished.

Our further investigation into the downstream targets of Foxos uncovered significantly reduced expression of apoptotic marker proteins such as p53, Bax, and Bim. Cyclin-dependent kinase inhibitors such as p27 and p21 are transcriptional targets of Foxos, and we observed marked reductions in these molecules (FIGS. 5A-B). Foxos are known to have functional interactions with other cell-proliferative markers such as Cdk2 and cyclin D1. These two molecules were also significantly increased in the Tet34-treated cells. These findings implicate that in addition to TGFβ activation, Tet34, but not Tet34s, promotes cellular survival likely via the Insulin/IGF-1/Foxo signaling.

Activation of insulin signaling pathway is particularly exciting in the adult because homeostasis of insulin signaling is critical for the modulation of different cellular processes such as cell survival, autophagy, and cell proliferation. In neurons, insulin is implicated in neurite outgrowth, axonal guidance, mitochondrial function, synaptic plasticity, and activity. Moreover, the insulin/IGF-1 pathway has also been shown to regulate the exit of neuroblasts from quiescence and promote hippocampal neurogenesis. Consistently, Foxos, the downstream transcription factors, are shown to regulate expression of many genes important for adult neurogenesis. For example, p21, p27, and p53 are transcriptional targets of Foxos and have vital roles in the maintenance of adult NSC quiescence. In Tg-CX3CL1-ct mice, enhanced neurogenesis may potentially be contributed from the increased levels of pFoxo1 and pFoxo3, in addition to the TGFB/Smad signaling discussed in our previous studies.

Neuronal apoptosis is associated with neurogenesis, physiologic aging and neurodegenerative conditions. Even during neurogenesis, the majority (50%-80%) of newborn neurons undergo apoptosis in the process of pruning, establishing neural circuits, and electrical activity. IGF-1 has been known to suppress apoptosis. Apart from p53, we observed significant attenuation of two other apoptotic markers, Bax and Bim, which are integral members of programmed cell death (apoptosis) and are critical for neurogenesis-associated apoptosis. Both Bax and Bim are transcriptional targets of Foxos. We inferred that downregulated Bax and Bim may contribute to reduced neuronal loss in 5xFAD mice expressing CX3CL1-ct.

In AD brains, dysregulated insulin/IGF-1 signaling has been found to contribute to neuroinflammation and oxidative stress, and insulin signaling dysfunction may underlie disease progression. Therapy targeting the insulin signaling pathway is viewed as a promising approach for treating neurodegenerative diseases. Clinical trials with insulin therapy face challenges due to concerns of off-target effects, limited penetration to the affected areas and the potential for development of insulin resistance. The ability of CX3CL1-ICD to robustly induce pro-survival and anti-apoptotic insulin/Foxo signaling pathways, demonstrated by the activation of their downstream molecules, mitigates the concern of insulin resistance in aging brains. While not wishing to be bound by theory, it is understood that Tet34 may provide dual ability to replenish neuronal loss and confer neuronal protection in various neurodegenerative diseases.

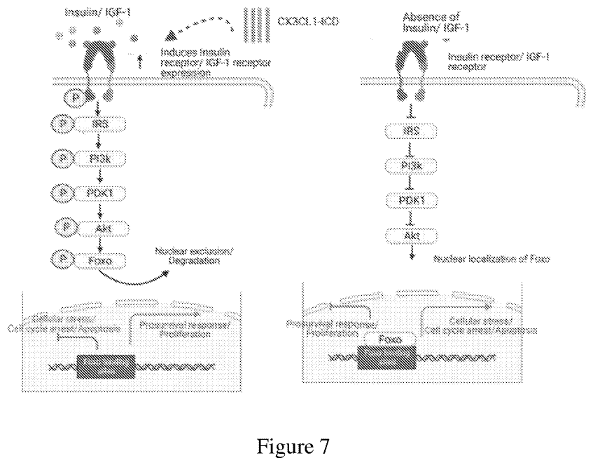

As shown in FIG. 7, when insulin or insulin growth factor-1 (IGF) bind to their cognate receptor, the receptor will be activated via the phosphorylation of its tyrosine residues within intracellular domain of β-subunit. The activated insulin/IGF-1 signaling pathway leads to activation of the downstream molecules including insulin receptor substrate-1 or -2 (IRS-1 and IRS-2), PI3K, PDK1, AKT1 and Foxo-1 or -3. Phosphorylated Foxo-1 or -3 will be retained in the cytosol while non-phosphorylated form of Foxo-1 or -3 will translocate into cell nucleus to induce pro-survival/proliferation and apoptotic responses. Tet34 appears to elevate IGF and its receptor levels via transcriptional regulation or posttranslational events as cells treated with CX3CL1-ICD peptides display increased Insulin receptor/IGF-1Rβ expression and activated insulin/IGF-1 signaling, indicating that CX3CL1-ICD has a function to activate these two receptors directly.

### Example 7: Determination of Whether Increased Expression of Intracellular C-Terminal CX3CL1 Domain (CX3CL1-ICD) has an Effect on the Development of Alzheimer's Neurofibrillary Tangles

Neuronal loss is particularly an age-associated event, which exacerbates the loss of synapses and causes even more severe cognitive dysfunction. Therapeutic intervention for AD treatment should not only reduce AD pathological hallmarks such as amyloid deposition and tau aggregation, but also mitigate synaptic impairment and neurodegeneration. Neuronal CX3CL1 has a novel function, which is able to reduce amyloid plaques in AD mice through its C-terminal domain (CX3CL1-ICD) (See, FIG. 16). In Tg-CX3CL1-ct/tTA mouse brains (under a tetracycline inducible promoter), released CX3CL1-ICD from overexpressed membrane-anchored CX3CL1-ct. In our previous study, we bred Tg-CX3C1-ct/tTA mice with 5xFAD mice, which develop amyloid plaques beginning at the age of 2 months and exhibit neuronal loss in layer V of cortex and subiculum at the age of 10 months. We found reduced amyloid deposition in Tg-CX3CL1-ct/tTA/5xFAD mice (reduction by about 23% in subiculum and 45% in cortical region, N=6) compared to 5xFAD littermates. Neuronal loss was also significantly reduced in Tg-CX3CL1-ct/tTA/5xFAD mice compared to control 5xFAD littermates. How CX3CL1-ICD reduces amyloid deposition was not yet mechanistically understood in that study. It should be noted that this original study was conducted in mice with constitutive expression of CX3CL1-ct [without suppression of transgene by Doxcycline (Dox) treatment]. For explore translational potential of CX3CL1, we intend to turn on the gene expression in the adult age and avoid the contribution through the developmental stage. Here we decide to investigate whether CX3CL1-ICD has an effect on the Tau pathology.

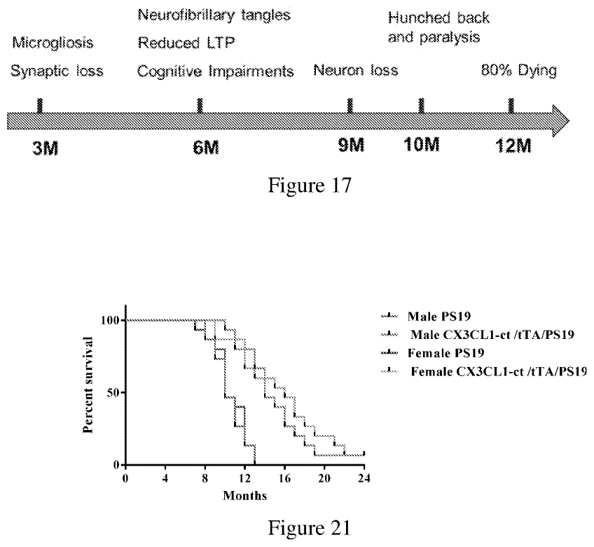

We then bred Tg-CX3CL1-ct mice with PS19 mice. PS19 transgenic mice (P301S Tg mice), generated by overexpressing the P301S tau by the Prp promoter, develop filamentous tau lesions at 6 months of age and exhibit brain atrophy due to neurodegeneration starting between eight to nine months of age (FIG. 17).

Mice were treated with Doxcycline (Dox), a more stable form of tetracycline, to suppress expression of CX3CL1-ct during the embryonic and early growth stages. Dox was given in the drinking water. To turn on the CX3CL1-ct expression, Dox would be removed from the drink water, and expression of CX3CL1 would be induced and could be detectable a few days after the removal of Dox in the drinking water.

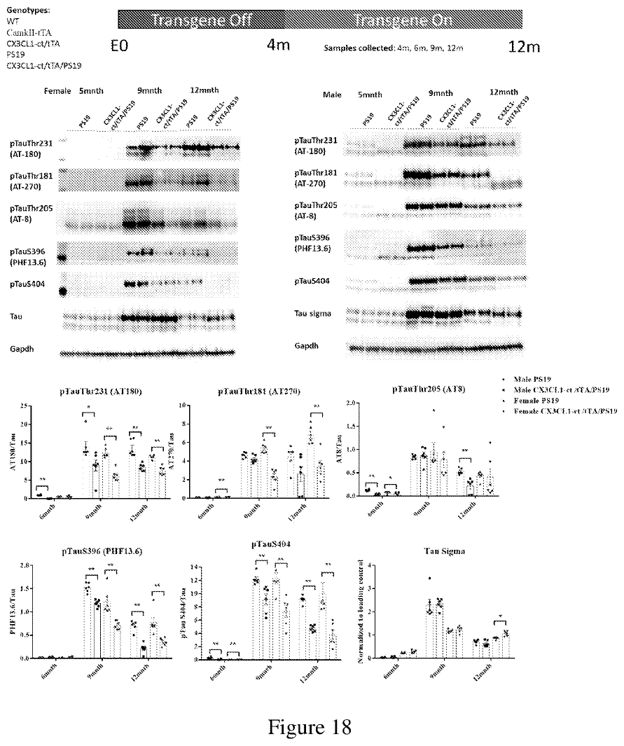

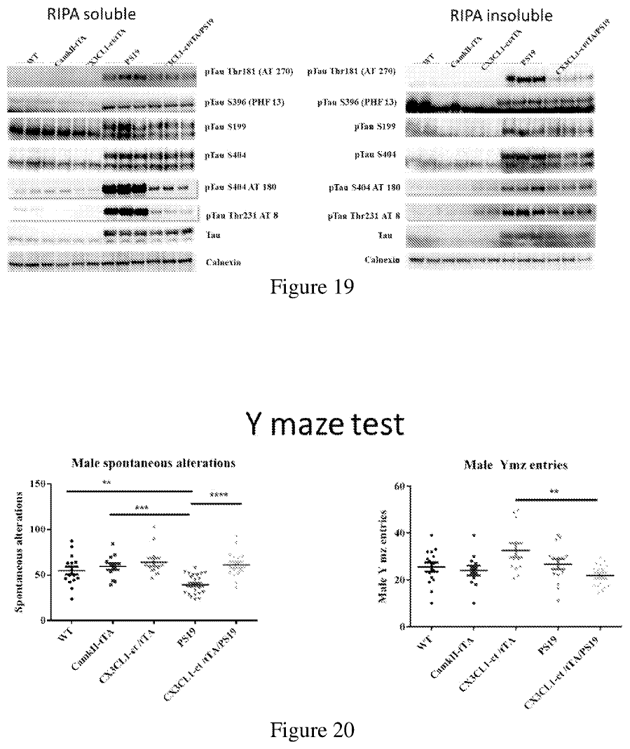

As shown in FIGS. 18-21, the released CX3CL1-ICD, through the expression of CX3CL1-ct, in mouse neurons is capable of suppressing formation of Tau aggregation through reduction of phosphorylation. The decrease in phosphorylated Tau level might be contributing to the improved cognitive function. PS19 mice can survive much longer when CX3CL1-ct gene is expressed. Together with published results, we show that CX3CL1-ICD will not only reduce amyloid plaques but also reduce tau aggregations in AD brains.

Method: PS19 transgenic mouse were crossed with CX3CL1-ct/tTA mouse to obtain CX3CL1-ct/tTA/PS19 mouse. The animals were given doxycycline via drinking water (0.05% doxycycline with 2% sucrose) from mating stage till P120 to suppress the expression of CX3CL1-ct gene under the CamkII-tTA promoter. The expression of CX3CL1-ct gene was turned on from P120 by withdrawal of doxycycline from drinking water. The animals were then sacrificed at regular intervals (4M, 6M, 9M, 12M) to collect the brain for protein analysis. Behavior assays were conducted at the age of 10 months by Y maze to evaluate any changes in special learning memory. A subset of animals was maintained till 24m to evaluate any changes in the survival pattern. For protein analysis by western blotting, protein fractions were prepared with RIPA buffer as mentioned. The denatured protein samples were run on 4-12% Bis-Tris gels prior transfer on 0.2 μm nitrocellulose membrane.

### Materials and Methods

Synthesis of tagged CX3CL1-ICD peptides and treatments: The peptide named as Tet34 has the sequence of RKMAGEMAEGLRYIPRSCGSNSYVLVPV (SEQ ID NO: 1) derived from the CX3CL1 C-terminus, while the Tet sequence for binding to neuron specific GT1b receptor HLNILSTLWKYRC (SEQ ID NO: 2) was fused to its N-terminus to provide HLNILSTLWKYRC RKMAGEMAEGLRYIPRSCGSNSYVLVPV (SEQ ID NO: 3). Tet34s has the same GT1b receptor binding sequence, but the remaining sequence has a scrambled order [HLNILSTLWKYRCGASNVYPIMKRRRYEASLVLGPGMECSV; SEQ ID NO: 4]. A batch of peptides were also custom-tagged with Alexa Fluor®-488 to track the kinetics of cellular and nuclear uptake (Thermo Fisher Scientific). Peptides were custom-synthesized (GenScript USA) and solubilized in 1× phosphate-buffered saline (PBS) to attain a working solution concentration of 2 μg/μl. Multiple aliquots were prepared to avoid repeated freeze/thaw cycles and were stored at −80° C. before use. Monolayers of N2A cells were grown to 70% confluence in either 2-well chamber slides (Thermo Fisher Scientific) or 100×20-mm tissue culture-treated dishes (Corning) before treatment with peptides. Cells were treated with 50 nM and 2 μM of peptides in media containing 2% fetal bovine serum for immunostaining and protein analysis, respectively. Post-peptide treatment, cells were incubated at 37° C., 5% CO₂ for different time points before analysis.

Treatment of primary hippocampal cultures with Tet-CX3CL1-ICD peptides: Wild type C57BL/6J E16.5 mouse embryos were euthanized and brains were placed in cold D-PBS. The hippocampi were removed, washed with HBSS, and digested in sterile-filtered papain (1 mg/mL) diluted in Neurobasal media (Gibco: 21103049) for 20 minutes. Tissues were triturated and digestion was halted with 10% fetal bovine serum and 1% glutamine in Neurobasal media and filtered through a 70 uM cell strainer. Cells were counted in trypan blue on a hemocytometer and 100,000 hippocampal cells were plated onto each chamber slide. 2-well chamber slides were previously coated with Poly-D-Lysine (0.1 mg/mL) for 24 hours at room temperature followed by washing with ddH₂O before plating. Media was exchanged after 24 hours with 2% B27-supplement (ThermoFisher: 17504044) and 1% Glutamine in Neurobasal media. Due to omission of cytarabine (AraC) from culture media, glial cells were allowed to proliferate resulting in a mixed culture. After 12 DIV, cells were treated with 2.5 nM Alexa Fluor®-488-conjugated Tet34 CX3CL1-ICD peptide or Tet34-scrambled for 12 hours. Cells were fixed in cold 100% methanol, permeabilized with 0.2% triton and blocked in 6% normal goat serum. Slides were incubated in rabbit anti-MAP2 (Millipore: AB5622), rat anti-GFAP (ThermoFisher: 13-0300), rabbit anti-Iba1 (Wako: 019-19741), and rabbit anti-Olig2 (Millipore: AB9610) primary antibodies overnight and incubated in Alexa Fluor® secondary antibodies for 2 hours at room temperature. Images were taken on a Zeiss confocal microscope using 63× and 20× objectives. Scale bar=20 um (63×) and 50 um (20×). Co-immunofluorescence quantification was conducted by measuring 488-pixel intensity within Map2-, Gfap-, and Iba1-positive cell bodies using ImageJ.

Cell proliferation assay: N2A cells were grown in 96-well tissue plates and treated with different concentrations of peptides for 24 hrs. Cell proliferation assays were performed using Click-iT™ EdU proliferation assay kits (Thermofisher C10499) according to the manufacturer's instructions. Briefly, cells were seeded overnight, followed by addition of different concentrations of peptides. The EdU was added to the media after 6 hrs of peptide treatment. The assay was terminated at 36 hrs, when Edu incorporation was measured with a microplate reader at 568 nm excitation and 585 nm emission wavelength.

Tg-CX3CL1-ct transgenic mice: The generation of Tg-CX3CL1-ct mice C-terminal fragment-derived CX3CL1 has been previously described in the art. The Tg-CX3CL1-ct pups were genotyped by PCR primers (Forward 5′CCGATATCTCTGTCGTGGCT 3′ (SEQ ID NO: 5) and Reverse 5′ GTTCCTCAGCCTTAGGGGTC 3′ (SEQ ID NO: 6)). Tg-CX3CL1-ct mice were bred with CaMKIIα-tTA mice (The Jackson Laboratory; 007004) to obtain Tg-CX3CL1-ct/tTA pups. Littermate CX3CL1-ct and CaMKIIα-tTA pups were used as controls. Mice were housed in designated animal rooms at 23° C. on a 12-h light/dark cycle with food and water available ad libitum. The doxycycline (Sigma-Aldrich) treatment was at 0.5 mg/ml in drinking water, supplemented with 2% sucrose and was administered to all animals used in experiment from E0 till P45. All experimental protocols were approved by the Institutional Animal Care and Use Committee of the UConn Health Center in compliance with the guidelines established by the Public Health Service Guide for the Care and Use of Laboratory Animals.

Western blotting and antibodies: Mouse brains were freshly dissected to isolate the hippocampus and cortex, and total proteins were extracted using modified radioimmunoprecipitation assay (RIPA) buffer. Routinely, at least two or three mice from each group were used for Western blot analysis. For in vitro studies, proteins were isolated from cells seeded in 6-well tissue culture-treated plates. Equal amounts of protein (50 μg) were resolved on a NuPAGE Bis-Tris gel (Invitrogen) and transferred onto a nitrocellulose membrane (Invitrogen). After protein transfer, the blot was incubated with the indicated antibodies. The sources of antibodies are: p-InsRß (Santa Cruz Biotechnology Cat #sc-81501, RRID: AB_1125643), InsRB (Cell Signaling Technology Cat #3020, RRID: AB_2249166), p-IGF-1Rb (Cell Signaling Technology Cat #3021, RRID: AB_331578), IGF-1Rβ (Cell Signaling Technology Cat #3027, RRID: AB_2122378), p-IRS1 (Thermofisher Cat #44816G, RRID: AB_2533768), IRS-1 (Cell Signaling Technology Cat #3407, RRID: AB_2127860),IRS-2 (Cell Signaling Technology Cat #4502, RRID: AB_2125774) p-Smad2 (#3104, RRID: AB_390732), Smad2/3 (#8685; RRID: AB_10889933), p-Smad1 (9553s; RRID: AB_2107775), Smad1 (#6944; RRID: AB_10860070), TGFß1 (SC146; RRID: AB_632486), TGFβ2 (SC-90; RRID: AB_2303237), TGFβ3 (SC-82; RRID: AB_2202303), pPI3K (Cell Signaling Technology Cat #17366, RRID: AB_2895293), PI3-kinase p85 (Cell Signaling Technology Cat #4257, RRID: AB_659889), pGSK3β (Cell Signaling Technology Cat #9336, RRID: AB_331405), Gsk3β (Cell Signaling Technology, Cat #9315, RRID: AB_490890), PARP (Cell Signaling Technology, Cat #9532, RRID: AB_659884), Cleaved Caspase 9 (Cell Signaling Technology, Cat #9509, RRID: AB_2073476), pFoxo1 (Cell Signaling Technology Cat #84192, RRID: AB_2800035), Foxo1 (Cell Signaling Technology Cat #2880, RRID: AB_2106495), pFoxo3 (Cell Signaling Technology Cat #13129, RRID: AB_2687495), Foxo3 (Cell Signaling Technology Cat #12829, RRID: AB_2636990), pPDK1 (Cell Signaling Technology Cat #9634, RRID: AB_2161307) (Cell Signaling Technology Cat #3438, RRID: AB_2161134), PDK1 (Cell Signaling Technology Cat #3062, RRID: AB_2236832), pAKT (Cell Signaling Technology Cat #13038, RRID: AB_2629447), AKT (Cell Signaling Technology Cat #4691, RRID: AB_915783), NeuroD1 (Abcam Cat #ab16508, RRID: AB_470254), ASCL1 (Santa Cruz Biotechnology Cat #sc-374104, RRID: AB_10918561), p53 (Santa Cruz Biotechnology Cat #sc-6243, RRID: AB_653753), p21 (Santa Cruz Biotechnology Cat #sc-6246, RRID: AB_628073), p27 (Cell Signaling Technology Cat #3688, RRID: AB_2077836), Bax (Santa Cruz Biotechnology Cat #sc-493, RRID: AB_2227995), Bim Cell Signaling Technology Cat #2933, RRID: AB_1030947), pSox2 (Cell Signaling Technology Cat #92186, RRID: AB_2800179), Sox2 (Millipore Cat #AB5603, RRID: AB_2286686), Sox5 (Abcam Cat #ab94396, RRID: AB_10859923), Sox8 (Santa Cruz Biotechnology Cat #sc-374446, RRID: AB_10989367), Sox9 (Cell Signaling Technology Cat #82630, RRID: AB_2665492), HA (#1867423; RRID: AB_10094468; Roche); and CX3CL1 (SC7225, RRID: AB_2087136; Santa Cruz). HRP-conjugated secondary antibodies were used and visualized using enhanced chemiluminescence (Thermo Fisher Scientific).

Real Time PCR quantitation: Mouse brains were freshly dissected to isolate the hippocampus and cortex, and midbrain. Total RNA was extracted using Trizol® (Thermofisher, Cat #15596026) according to the manufacturer's protocol. 4 μg of total RNA was used to perform cDNA transcription with High capacity cDNA Reverse Transcription kit (Thermofisher, Cat #4368813). 2 μl of cDNA was used for real time PCR quantitation of IGF-1Rβ mRNA, IGF1 mRNA and 18S rRNA with SYBR® Green Master Mix (Thermofisher, Cat #A25742). The qPCR primers used were IGF-1Rβ (Forward 5′ GTG GGG GCT CGT GTT TCT C 3′ (SEQ ID NO: 7) and Reverse 5′ GAT CAC CGT GCA GTT TTC CA 3′ (SEQ ID NO: 8)), IGF1 (Forward 5′ 5′-CCG AGG GGC TTT TAC TTC AAC AA3′ (SEQ ID NO: 9) and Reverse 5′ 5′CGG AAG CAA CAC TCA TCC ACA A 3′ (SEQ ID NO:10)) and 18S rRNA (Forward 5′ TGTGCCGCTAGAGGTGAAATT 3′ (SEQ ID NO: 11) and Reverse 5′ TGGCAAATGCTTTCGCTTT 3′ (SEQ ID NO: 12)).

Amyloid-β treatment of cultured cells: N2A cell monolayers at 70% confluency were treated with 5 μM Tet-peptides for 24 hrs. Reconstituted human Aβ₁₋₄₂ (Biosource) was added to the cells at a 1 μM concentration and incubated for 12 hrs. The Aβ₁₋₄₂ was reconstituted according to the manufacturer's protocol. Briefly, the lyophilized peptide was dissolved in HPLC water followed by dilution in PBS. The solution was incubated at 37° C., shaker for 24 hrs to obtain the neurotoxic form of Aβ₁₋₄₂. In our experience, we get approximately 70% oligomeric Aβ₁₋₄₂ with this protocol. Total cell lysate was collected by RIPA lysis buffer for western blot evaluation of apoptotic pathways.

Hydrogen peroxide induced apoptosis in cultured N2A cells: N2A cell monolayers grown to 70% confluency were treated with 2 μM Tet-peptides for 24 hrs in 2% serum for signaling induction. The cells were then incubated with 50 μM Hydrogen Peroxide (Sigma, #H1009) for different time points to induce apoptosis. A batch of cells were harvested at 1 hr to evaluate apoptosis by western blots. Flow cytometry using Alexa Fluor®-488 Annexin V kit (Thermofisher Scientific #V13241) was used to determine changes in the population of cells undergoing apoptosis at different time points of treatment.

Akt Inhibition assay in cultured N2A cells: N2A cell monolayers at 70% confluency were treated with 2 μM Tet-peptides for 24 hrs in 2% serum for signaling induction. The cells were then incubated with 2 μM MK2206, Akt inhibitor (Selleckchem #S1078) for 3hrs prior to total cell lysate collection. Western blot was performed to evaluate the specificity of Tet-peptides in the induction of IGF1Rb/Akt/Foxo signaling pathway.

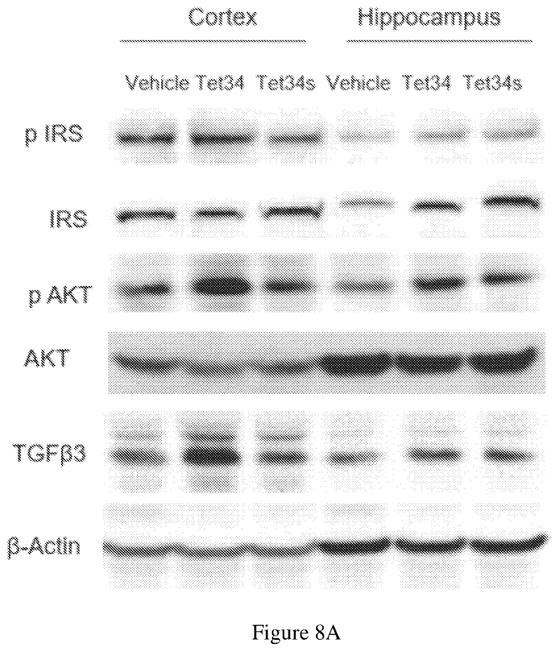

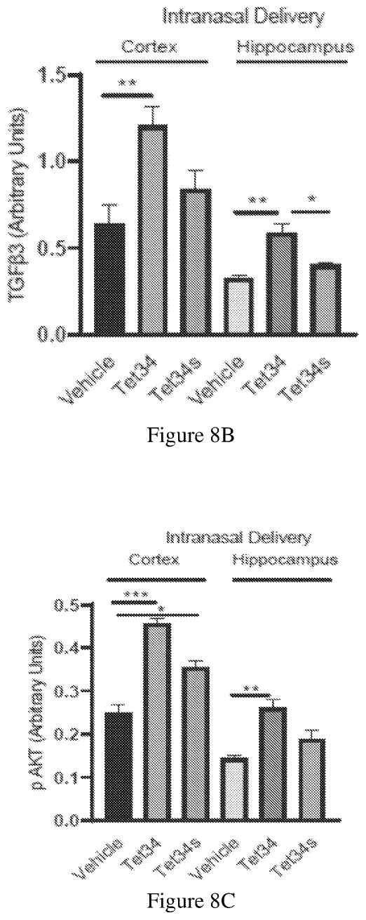

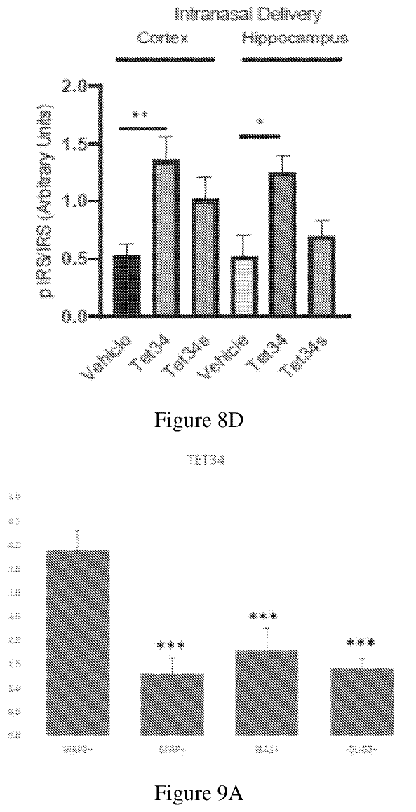

Intranasal Tet34/Tet34s (2 μM) delivery 24 hour post intranasal delivery: To examine whether CX3CL1-ct can be delivered to the brain via a non-invasive intranasal route, the C57BL/6 mice were immobilized by anesthetizing mice with intraperitoneal ketamine/Xylazine injection. Post anesthesia induction, Tet34 peptide (2 μM) mimicking Cx3cl1-intracellular domain was instilled intranasal, while the other sets of mice received scrambled control Tet34s peptide as well as vehicle control phosphate buffer saline. Twenty-four hours post-delivery, the brains were harvested and processed for examining the ability of Tet34 to initiate neuronal signaling. Cortex and hippocampal brain lysate were immunoprobed for some of the signaling molecules including pIRS, pAKT and TGFβ3) which were previously identified via in-vitro neuronal cell culture studies post TET34 treatment. As compared to scrambled control or vehicle, our western blot analysis revealed significant upregulation in the phosphorylation of insulin receptor (pIRS) and AKT (PAKT) as well as TGFβ3 in both hippocampus and cortex of the mice treated with a single dose of Tet34 peptide suggesting that TET34 peptide can be efficiently delivered in the brain and induces signaling pathways commonly associated with neurogenesis (FIGS. 8A-D).

Statistical analysis: Quantitative data are presented as mean±SEM. All experiments were independently repeated at least three times. Statistical analyses were conducted using Prism 6 software (GraphPad Software). Statistical comparisons between groups were analyzed for significance by one-way analysis of variance (ANOVA) with Tukey's post hoc test and Student's t test. Significant P values are denoted by asterisks in the text and figures (*P<0.05, **P<0.01, ***P<0.001). Error bars in each case represent standard error of the mean.

### DEFINITIONS

Compounds and materials are described using standard nomenclature. Unless defined otherwise, all technical and scientific terms used herein have the same meaning as is commonly understood by one of skill in the art to which this invention belongs. The following terms are used to describe the invention of the present disclosure. In instances where a term is not specifically defined herein, that term is given an art-recognized meaning by those of ordinary skill applying that term in context to its use in describing the present disclosure.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one clement selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total pharmaceutical composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a pharmaceutical composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “administering” means the actual physical introduction of a pharmaceutical composition into or onto (as appropriate) a subject, a host or cell. Any and all methods of introducing the pharmaceutical composition into the subject, host or cell are contemplated according to the invention; the method is not dependent on any particular means of introduction and is not to be so construed. Means of introduction are well-known to those skilled in the art, and also are exemplified herein.

As used herein, “optional” or “optionally” means that the subsequently described event or circumstance may or may not occur, and that the description includes instances where said event or circumstance occurs and instances where it does not.

As used herein, the term “pharmaceutically acceptable” refers to pharmaceutical compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the terms “treat,” “treating,” and “treatment” include inhibiting the pathological condition, disorder, or disease, e.g., arresting or reducing the development of the pathological condition, disorder, or disease or its clinical symptom or symptoms; or relieving the pathological condition, disorder, or discase, e.g., causing regression of the pathological condition, disorder, or disease or its clinical symptom or symptoms. These terms also encompass therapy and cure. Treatment means any way the symptom or symptoms of a pathological condition, disorder, or disease are ameliorated or otherwise beneficially altered. Preferably, the subject in need of such treatment is a mammal, preferably a human.

As used herein, the term “effective amount” refers to the amount of a therapy, which is sufficient to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent). An effective amount can require more than one dose.

Effective amounts may vary depending upon the biological effect desired in the individual, condition to be treated, and/or the specific characteristics of the pharmaceutical composition according to the present invention and the individual. In this respect, any suitable dose of the pharmaceutical composition can be administered to the patient (e.g., human), according to the type of disease to be treated. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the pharmaceutical composition according to the present invention desirably comprises about 0.1 mg per kilogram (kg) of the body weight of the patient (mg/kg) to about 400 mg/kg (e.g., about 0.75 mg/kg, about 5 mg/kg, about 30 mg/kg, about 75 mg/kg, about 100 mg/kg, about 200 mg/kg, or about 300 mg/kg). In another embodiment, the dose of the pharmaceutical composition according to the present invention comprises about 0.5 mg/kg to about 300 mg/kg (e.g., about 0.75 mg/kg, about 5 mg/kg, about 50 mg/kg, about 100 mg/kg, or about 200 mg/kg), about 10 mg/kg to about 200 mg/kg (e.g., about 25 mg/kg, about 75 mg/kg, or about 150 mg/kg), or about 50 mg/kg to about 100 mg/kg (e.g., about 60 mg/kg, about 70 mg/kg, or about 90 mg/kg).

The term “subject” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject is a human, such as a human being treated or assessed for a disease, disorder or condition, a human at risk for a disease, disorder or condition, a human having a disease, disorder or condition, and/or human being treated for a discase, disorder or condition as described herein. In some embodiments, the subject does not suffer from an ongoing autoimmune disease. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this invention. In addition, ranges of values using a combination of any of the above-recited values as upper and/or lower limits are intended to be included.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed clement as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

While the invention has been described with reference to an exemplary embodiment, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A polypeptide comprising an amino acid sequence that is at least 90% identical to SEQ ID NO: 1.

2. The polypeptide of claim 1, wherein the polypeptide comprises an amino acid sequence at least 95% identical to SEQ D NO: 1.

3. The polypeptide of claim 1, wherein the polypeptide comprises an amino acid sequence 100% identical to SEQ ID NO: 1.

4. The polypeptide of claim 1, wherein the polypeptide further comprises a heterologous peptide, a detectable label, or both.

5. The polypeptide of claim 4, wherein the heterologous peptide is a signal peptide comprising a neuron targeting tag.

6. The polypeptide of claim 5, wherein the neuron targeting tag comprises SEQ ID NO: 2.

7. The polypeptide of claim 1, wherein the polypeptide is a synthetic polypeptide or an isolated polypeptide.

8. A pharmaceutical composition comprising the polypeptide of claim 1 and a pharmaceutically acceptable excipient.

9. A nucleic acid expressing the polypeptide of claim 1.

10. An expression cassette comprising the nucleic acid of claim 9.

11. A vector comprising the expression cassette of claim 10.

12. A host cell comprising the vector of claim 11.

13. A method of treating a subject exhibiting a symptom of a neurodegenerative disease and/or diagnosed with a neurodegenerative disease, comprising administering to the subject the pharmaceutical composition of claim 8.

14. The method of claim 13, wherein the neurodegenerative disease is Alzheimer's disease, Parkinson's disease, amyotrophic lateral sclerosis, ataxia such as Friedreich ataxia, Huntington's disease, Lewy body disease, spinal muscular atrophy, multiple system atrophy, motor neuron disease, progressive supranuclear palsy (PSP), corticobasal syndrome (CBS), frontal dementia diseases, or a combination thereof.

15. The method of claim 13, wherein the symptom of a neurodegenerative disease comprises confusion, disinhibition, apathy, anxiety, memory loss, difficulty thinking or concentrating, behavior changes, mood changes, depression, delusions, hallucinations, tingling or numbness, pain, muscle spasms, weakness, paralysis, coordination issues, fatigue, slowed movements, shaking, tremors, balance problems, shuffling steps, hunched posture, loss of muscle control, weakness, paralysis, or a combination thereof.

16. The method of claim 13, wherein the subject is a human subject.

17. A method of treating a subject exhibiting a symptom of Type II diabetes and/or diagnosed with Type II diabetes comprising administering to the subject the pharmaceutical composition of claim 8.

18. The method of claim 17 wherein the symptom of Type II diabetes is frequent urination, excessive thirst, fatigue, unexpected weight loss, thrush, slow healing, blurred vision, increased hunger, or a combination thereof.

19. A method of treating a subject exhibiting a symptom of aging. the method comprising administering to the subject the pharmaceutical composition of claim 8.

20. The method of claim 17, wherein the subject is a human subject.


SEQUENCE LISTING (12 sequences)

SEQ ID NO: 1 - protein, 28 aa, Homo sapiens
  source 1..28: note = intracellular domain of CX3CL1
RKMAGEMAEGLRYIPRSCGSNSYVLVPV

SEQ ID NO: 2 - protein, 13 aa, Clostridium tetani
HLNILSTLWKYRC

SEQ ID NO: 3 - protein, 41 aa, synthetic construct
  source 1..41: note = AA 1..13 N-terminal tag from tetanus toxin
HLNILSTLWKYRCRKMAGEMAEGLRYIPRSCGSNSYVLVPV

SEQ ID NO: 4 - protein, 41 aa, synthetic construct
  source 1..41: note = AA 1..13 N-terminal tag from tetanus toxin
HLNILSTLWKYRCGASNVYPIMKRRRYEASLVLGPGMECSV

SEQ ID NO: 5 - DNA, 20 bp, synthetic construct
  source 1..20: mol_type = other DNA; note = Primer
ccgatatctctgtcgtggct

SEQ ID NO: 6 - DNA, 20 bp, synthetic construct
  source 1..20: mol_type = other DNA; note = Primer
gttcctcagccttaggggtc

SEQ ID NO: 7 - DNA, 19 bp, synthetic construct
  source 1..19: mol_type = other DNA; note = Primer
gtgggggctcgtgtttctc

SEQ ID NO: 8 - DNA, 20 bp, synthetic construct
  source 1..20: mol_type = other DNA; note = Primer
gatcaccgtgcagttttcca

SEQ ID NO: 9 - DNA, 23 bp, synthetic construct
  source 1..23: mol_type = other DNA; note = Primer
ccgaggggcttttacttcaacaa

SEQ ID NO: 10 - DNA, 22 bp, synthetic construct
  source 1..22: mol_type = other DNA; note = Primer
cggaagcaacactcatccacaa

SEQ ID NO: 11 - DNA, 21 bp, synthetic construct
  source 1..21: mol_type = other DNA; note = Primer
tgtgccgctagaggtgaaatt

SEQ ID NO: 12 - DNA, 19 bp, synthetic construct
  source 1..19: mol_type = other DNA; note = Primer
tggcaaatgctttcgcttt